(19) United States
(12) Patent Application Publication
(10) Pub. No.: US 2016/0030513 A1
(43) Pub. Date: Feb. 4, 2016

(54) Notch1 Decoy Antagonists Protect From Obesity-Induced Insulin Resistance and Fatty Liver

(71) Applicants: Utpal Pajvani, Leonia, NJ (US); Jan Kitajewski, Ridgewood, NJ (US)

(72) Inventors: Utpal Pajvani, Leonia, NJ (US); Jan Kitajewski, Ridgewood, NJ (US)

(73) Assignee: The Trustees of Columbia University in the City of New York, New York, NY (US)

(21) Appl. No.: 14/814,407

(22) Filed: Jul. 30, 2015

Related U.S. Application Data
(63) Continuation-in-part of application No. PCT/US2014/026717, filed on Mar. 13, 2014.
(60) Provisional application No. 61/800,180, filed on Mar. 15, 2013.
(60) Provisional application No. 62/031,090, filed on Jul. 30, 2014.

(51) Int. Cl. A61K 38/17; A61K 45/06

(52) U.S. Cl. CPC A61K 38/177; A61K 45/06

(57) ABSTRACT

The present invention provides a method of treating a subject suffering from a fatty liver disease which comprises administering to the subject a Notch decoy protein or Jagged inhibitor in an amount effective to treat the subject's fatty liver disease.

The present invention provides a composition comprising a pharmaceutically acceptable carrier and an amount of a Notch decoy protein or Jagged inhibitor effective to treat a fatty liver disease.

The present invention provides a package comprising:

(a) the pharmaceutical composition of the invention and
(b) instructions for using the pharmaceutical composition of step (a) to treat the fatty liver disease.

This application is (a) a continuation-in-part of PCT International Application No. PCT/US2014/026717, filed on Mar. 13, 2014, claiming priority of U.S. Provisional Application No. 61/800,180, filed Mar. 15, 2013 and (b) claims priority of U.S. Provisional Application No. 62/031,090, filed Jul. 30, 2014, the content of each of the foregoing applications is hereby incorporated by reference in its entirety.

Throughout this application, various publications are referenced. Full citations for these publications may be found at the end of the specification or at the end of each experimental section. The disclosures of these publications are hereby incorporated by reference into this application to describe more fully the art to which this invention pertains.

### BACKGROUND OF THE INVENTION

Metabolic diseases in their protean incarnations are likely to define health, public policy, and economics of the 21st century (Wang 2011). Aside from surgical remediation, progress in their treatment with lifestyle or pharmacologic therapies has been disappointing.

Altered insulin signaling is often associated with excessive hepatic triglyceride content (hepatosteatosis), a correlate of hepatic failure, hepatocellular cancer and need for liver transplantation (Savage 2010). Activation of the nutrient-sensing mTor pathway stimulates hepatic de novo lipogenesis, providing not only an explanation for how nutrient excess translates into elevated hepatic fat, but also for the apparent paradox whereby increased Akt—an mTor substrate—can simultaneously promote insulin sensitivity and hepatosteatosis.³ Thus, treatment of hepatocytes with rapamycin, an allosteric inhibitor of the mTorc1-dependent functions of mTor, prevents insulin activation of the lipogenic transcription factor Srepb1c (Li 2010; Sabatini 2006). Although interpretation of in vivo studies in rodents chronically treated with rapamycin, and clinical experience in rapamycin-treated patients, is clouded by their effects to disrupt insulin signaling in other tissues, mice with disruptions in hepatic mTor signaling have offered insight into the convergence of mTor and insulin pathways in the combined regulation of glucose and lipid metabolism (Blattler 2012; Lamming 2012; Houde 2010; Howell 2011). Liver-specific knockout of either the critical mTorc1 component Raptor, or the mTorc2-defining subunit Rictor, protect from diet-induced hepatic steatosis, likely due to reduced lipogenesis (Peterson 2011; Hagiwara 2012). Interestingly, hepatocyte-specific knockout of the mTor inhibitor, Tsc1, activates mTorc1 signaling and protects from diet-induced fatty liver due to effects on Insig2a, a regulator of Srebp1c function, suggesting that tight regulation of this pathway is physiologically relevant (Yecies 2011).

The bifurcation of the insulin signaling pathways after Akt—to FoxO1 for glucose production, and to mTor/Srebp1c for lipogenesis—raises the question of whether these pathways have additional inputs. Notch signaling is critical for cell type specification and lineage restriction (Bolos 2007). Cell surface-tethered ligands (Jagged and Delta-like) bind Notch receptors on neighboring cells, resulting in a series of cleavage events that culminate in γ-secretase-dependent liberation of the Notch intracellular domain (NICD) (Fortini 2009). NICD translocates to the nucleus, where it binds to and co-activates the transcriptional effector Rbp-Jk, promoting expression of the Hairy enhancer of split (Hes) and Hes-related (Hey) family of genes (Dufraine 2008). Homozygous null alleles of components of this signaling pathway result in embryonic lethality, demonstrating their importance to normal development (Swiatek 1994; Oka 1995; Shen 1997). Importantly, Notch signaling is therapeutically accessible, and inhibitors are in advanced clinical development for cancer (Rizzo 2008).

Fatty liver disease is a condition where large vacuoles of triglyceride fat accumulate in liver cells via the process of abnormal retention of lipids. Despite having multiple causes, fatty liver can be considered a single disease that occurs worldwide in those with excessive alcohol intake and those who are obese and is diagnosed as either alcoholic fatty liver disease or non-alcoholic fatty liver disease (Bayard 2006).

Alcoholic liver disease is the major cause of liver disease in western countries. Non-alcoholic fatty liver disease is the leading cause of elevated liver enzyme levels in U.S. adults and is the most common cause of cirrhosis which cannot be explained by hepatitis, alcohol abuse, toxin exposure, autoimmune disease, congenital liver disease, vascular outflow obstruction, or biliary tract disease (Bayard 2006).

Despite recent advancements in treatment, there exists a need for safe and effective treatments for fatty liver disease.

### SUMMARY OF THE INVENTION

The present invention provides a method of treating a subject suffering from a fatty liver disease which comprises administering to the subject a Notch decoy protein or Jagged inhibitory in an amount effective to treat the subject's fatty liver disease.

The present invention provides a composition comprising a pharmaceutically acceptable carrier and an amount of a Notch decoy protein effective to treat a fatty liver disease.

The present invention provides a package comprising:

(a) the pharmaceutical composition of the invention; and
(b) instructions for using the pharmaceutical composition of step (a) to treat the fatty liver disease.

### BRIEF DESCRIPTION OF THE FIGURES

FIG. 1A-FIG. 1J: Regulation of hepatic Notch activity. *p<0.05 vs. db/+ or wt mice. Data show means±SEM. FIG. 1A: Western blot of cleaved Notch1 receptor (NICD) in livers from fasted and refed 9-week-old, chow-fed C57/Bl6 mice (n=S/group). FIG. 1B Quantification of the data in FIG. 1A. FIG. 1C: Expression of insulin targets in livers from fasted and refed 9-week-old, chow-fed C57/Bl6 mice (n=5/group). FIG. 1D: Expression of Srebp1c targets in livers from fasted and refed 9-week-old, chow-fed C57/Bl6 mice (n=S/group). FIG. 1E: Expression of Notch targets in livers from fasted and refed 9-week-old, chow-fed C57/Bl6 mice (n=5/group). FIG. 1F: Regulation of Notch targets in 16-week-old L-Rbpj and control (Cre−) mice, fasted for 16-h or fasted for 16-h followed by 4-h refeeding (n=6/group). Fasted values are set arbitrarily at 1 for both groups. *p<0.05 vs. fasted mice. FIG. 1G Western blot of cleaved Notch1. FIG. 1H Notch target gene expression in livers from fasted, 16-week old chow-fed or high-fat diet (HFD)-fed mice (n=12/group). *p<0.05 vs. chow-fed mice. FIG. 1I: Notch target expression in livers from db/db or control (db/+) mice (n=5/group). FIG. 1J: Notch target expression in hepatocytes from ob/ob or control (wt) mice, all sacrificed in the ad libitum state (triplicate wells, representative of 2 individual experiments).

FIG. 2A-FIG. 2B: Nutritional regulation of hepatic Notch1. FIG. 2A: Western analysis of NICD in 8-week-old, chow-fed C57/BL6 male mice fasted for 24-h, and refed for the indicated times. FIG. 2B: Protein expression normalized to tubulin. Data show means±SEM.

FIG. 3A-3O: Decreased hepatic steatosis in HFD-fed L-Rbpj mice. *p<0.05, **p<0.01, ***p<0.001 vs. Cre− mice or hepatocytes. Data shown means±SEM. FIG. 3A: Body weight of Cre− and L-Rbpj male mice (n=6-8/group) on standard chow, or HFD started at weaning. FIG. 3B: H&E staining from age- and weight-matched L-Rbpj and Cre− mice on HFD. FIG. 3C: Hepatic lipids (inset shows expanded graph for cholesterol) in mice fed HFD from weaning and sacrificed at 20 weeks after a 16-h fast (n=8/group). FIG. 3D: Liver and epidydimal white adipose tissue (WAT) weight in mice fed HFD from weaning and sacrificed at 20 weeks after a 16-h fast (n=8/group). FIG. 3E: Triglyceride secretion in 12 week-old HFD-fed Cre− and L-Rbpj mice, fasted for 5-h, then injected with Poloxamer 407 (n=6/group). FIG. 3F: Fatty acid oxidation determined through 14CO2 release after incubation of primary hepatocytes from 16-week old Cre− and L-Rqpj mice with 14C-oleic acid (triplicate wells, representative of 2 individual experiments). FIG. 3G: Expression of genes regulating fatty acid oxidation in livers of 20-week old, HFD-fed mice sacrificed after 16-h fast, followed in some animals by 4-h refeeding (n=8/group). FIG. 3H: β-OH butyrate levels in 20-week-old, HFD-fed mice sacrificed after a 16-h fast. FIG. 3I: Hepatic lipogenic protein expression in livers from 20-week-old, HFD-fed mice sacrificed after a 16-h fast, followed by 6-h refeeding, measuring 3H2O incorporation into fatty acids (n=7/group). FIG. 3J: de novo lipogenesis in livers from 20-week-old, HFD-fed mice sacrificed after a 16-h fast, followed by 6-h refeeding, measuring 3H2O incorporation into fatty acids (n=7/group). FIG. 3K: De novo lipogenesis in insulin-treated hepatocytes from chow-fed 16-week-old mice, measuring 14C-acetate incorporation into fatty acids (triplicate wells, representative of 2 individual experiments). FIG. 3L: Basal and insulin-stimulated Srebp1c expression in primary hepatocytes from chow-fed, 16-week-old Cre− and L-Rbpj littermates (triplicate wells, representative of 2 individual experiments. FIG. 3N: Fasn-luciferase activity in primary hepatocytes from chow-fed, 16-week-old Cre− and L-Rbpj littermates (triplicate wells, representative of 2 individual experiments. FIG. 3N: Western blot analysis of Akt and mTor signaling in livers from HFD-fed mice sacrificed after a 5-hr fast. FIG. 3O: Western blots from hepatocytes isolated from 16-week-old mice, transduced with control or Notch1-IC adenovirus, treated with or without 10 nM insulin or 25 nM rapamycin for 4-h. Protein expression normalized to either actin or tubulin.

FIG. 4A-FIG. 4E: Metabolic analyses in L-Rbpj and L-Foxo1 mice. *p<0.05, **p<0.01, ***p<0.001 vs. Cre− mice. Data show means±SEM. FIG. 4A: Serum TG and cholesterol in HFD-fed mice after 16-h fast, or 2-h refeeding (n=6-8/group). FIG. 4B: non-esterified fatty acids (NEFA) in HFD-fed mice after 16-h fast, or 2-h refeeding (n=6-8/group). FIG. 4C: Relative hepatic triglyceride content in chow-fed, 12-week old L-Rbpj, L-Foxo1 and L-Rbpj/Foxo1 mice, normalized to respective Cre− littermates. FIG. 4D: Serum TG before (fasted) and following olive oil gavage (300 μl/mouse) in HFD-fed mice. FIG. 4E: Pparγ and PPARγ target (Ap2, Cd36) expression in primary hepatocytes (triplicate wells, representative of 2 individual experiments).

FIG. 5A-FIG. 5F: Metabolic effects of high-fat feeding in L-Rbpj mice. *p<0.05, **p<0.01, ***p<0.001 vs. Cre− mice. Data show means±SEM. FIG. 5A: Glucose in 5-h-fasted Cre− or L-Rbpj mice fed HFD for 3 weeks. FIG. 5B: insulin in 5-h-fasted Cre− or L-Rbpj mice fed HFD for 3 weeks. FIG. 5C: non-esterified fatty acids (NEFA) in 5-h-fasted Cre− or L-Rbpj mice fed HFD for 3 weeks. FIG. 5D: hepatic TG in 5-h-fasted Cre− or L-Rbpj mice fed HFD for 3 weeks. FIG. 5E: serum lipid levels in 5-h-fasted Cre− or L-Rbpj mice fed HFD for 3 weeks. FIG. 5F: β-OH butyrate levels in Cre− and L-Rbpj mice fed HFD for 3 weeks, fasted (16-h), or refed (4-h).

FIG. 6A-FIG. 6H: Notch decoy increases insulin sensitivity and decreases hepatic lipid content. Protein expression normalized to either Actin or Tubulin. Mice were 12-week-old male C57Bl/6, unless otherwise indicated. *p<0.05 vs. Fc. Data show means±SEM. FIG. 6A: Glucose 14 d after delivery of Notch1 decoy or Fc control adenovirus in HFD-fed mice, fasted for 16-h (n=6/group). FIG. 6B: insulin 14 d after delivery of Notch1 decoy or Fc control adenovirus in HFD-fed mice, fasted for 16-h (n=6/group). FIG. 6C: liver weight 14 d after delivery of Notch1 decoy or Fc control adenovirus in HFD-fed mice, fasted for 16-h (n=6/group). FIG. 6D: hepatic triglyceride 14 d after delivery of Notch1 decoy or Fc control adenovirus in HFD-fed mice, fasted for 16-h (n=6/group). FIG. 6E: Western blots of liver proteins from HFD-fed mice transduced with Notch decoy or Fc control adenovirus and fasted for 16-h. FIG. 6F: Expression of fatty acid oxidation genes in livers from fasted mice transduced with Notch decoy (n=6/group). FIG. 6G: Srebp1c levels in primary mouse hepatocytes transduced with Notch decoy or Fc adenovirus (triplicate wells, representative of 2 individual experiments). FIG. 6H: Western blots of liver protein from HFD-fed mice, sacrificed after a 16-h fast, transduced with Notch decoy or Fc control adenovirus.

FIG. 7A.-FIG. 7E: Effect of Notch decoy on weight, adiposity, and lipids. *p<0.05 vs. Fc. Data shown means±SEM. FIG. 7A: Body weight from fasted mice transduced with Notch decoy or Fc control. FIG. 7B: relative epidydimal white adipose tissue (eWAT) weight from fasted mice transduced with Notch decoy or Fc control. FIG. 7C: Serum TG measured under the conditions indicated 7-d after transduction of mice with Notch1 decoy or Fc adenovirus. FIG. 7D: cholesterol measured under the conditions indicated 7 days after transduction of mice with Notch1 decoy or Fc adenovirus. FIG. 7E: Pparγ and PPARγ target (Ap2, Cd36) expression in primary hepatocytes from 12-week-old mice, transduced with Notch decoy as compared to Fc control adenovirus (triplicate wells, representative of 2 individual experiments).

FIG. 8A-FIG. 8P: Activation of hepatic Notch increases mTor, lipogenic genes, and steatosis in chow-fed mice. *p<0.05, **p<0.01, ***p<0.001 vs. Fc or GFP. Protein expression normalized to either Actin or Tubulin. Mice were 8-week-old C57Bl/6 males, unless otherwise indicated. Data show means±SEM. FIG. 8A: Oil-Red-O staining in livers of 16-h fasted mice (inset shows expanded graph for cholesterol) 7-d after adenoviral delivery of GFP (control) or constitutively active Notch1 (N1-IC) (n=6/group). FIG. 8B: weight in livers of 16-h fasted mice (inset shows expanded graph for cholesterol) 7-d after adenoviral delivery of GFP (control) or constitutively active Notch1 (N1-IC) (n=6/group). FIG. 8C: lipid content in livers of 16-h fasted mice (inset shows expanded graph for cholesterol) 7-d after adenoviral delivery of GFP (control) or constitutively active Notch1 (N1-IC) (n=6/group). FIG. 8D: Liver Western blots in mice transduced with GFP or N1-IC adenovirus, sacrificed after a 16-h fast followed by 2-h refeeding. (n=6/group). FIG. 8E: Gene expression analysis in mice transduced with GFP or N1-IC adenovirus, sacrificed after a 16-h fast followed by 2-h refeeding. (n=6/group). FIG. 8F: De novo lipogenesis in hepatocytes after transduction with GFP or N1-IC adenovirus and incubation with 10 nM insulin (triplicate wells, representative of 2 individual experiments). FIG. 8G: Hepatic triglyceride content 7-d after GFP or N1-IC transduction in 16-h fasted, 24-week-old, chow-fed Cre− and L-Rbpj mice. FIG. 8H: Gene expression after GFP or N1-IC transduction of primary hepatocytes from Cre− and L-Rbpj littermates, followed by incubation with 10 nM insulin (triplicate wells, representative of 2 individual experiments). *p<0.05 vs. untreated cells, &p<0.05 vs. insulin-treated cells, *p<0.05 vs. N1-IC transduced, insulin-treated cells. FIG. 8I: De novo lipogenesis in isolated hepatocytes after transduction with GFP (arbitrarily set to a value of 1) or N1-IC and incubation with 10 nM insulin (triplicate wells, representative of 2 individual experiments). FIG. 8J: Western blot from livers of mice transduced with GFP or N1-IC, fasted for 16-h, or refed for 2-h. FIG. 8K-FIG. 8M: Quantitation of the phospho/total mTor, 4E-BP1 and S6k levels from the experiments in FIG. 8J. FIG. 8N: Western blot from FAQ hepatoma cells transduced with Fc (−) or N1-IC (Notch1), incubated in serum-free and amino acid-free medium for 4-h, followed by treatment with 10 nM insulin or 4× amino acid mixture for 4-h. FIG. 8O: Western blot in primary hepatocytes transduced with Fc or N1-IC, after treatment with 10 nM insulin and/or 25 nM rapamycin. FIG. 8P: Fasn-luciferase assays in FAD hepatoma cells transduced with N1-IC, N1-decoy or Fc (control) adenovirus and treated with 10 nM insulin.

FIG. 9A-FIG. 9F: mTor inhibition prevents Notch-induced fatty liver. Data shown means±SEM. FIG. 9A: Fasn-luciferase in FAO hepatoma cells transfected with either Raptor or scrambled (scr) shRNA, transduced with either Fc (−) or N1-IC (Notch) adenovirus and treated for 16-h with 100 nM insulin. ***p<0.001 vs. Fc control, &p<0.001 vs. scrambled shRNA, *p<0.001 vs. no insulin. FIG. 9B Gene expression in primary hepatocytes after transduction with GFP (−) or N1-IC (Notch) adenovirus, followed by incubation with 10 nM insulin and/or 25 nM rapamycin (triplicate wells, representative of 2 individual experiments). *p<0.05 vs. untreated cells, &p<0.05 vs. insulin-treated cells, *p<0.05 vs. N1-IC transduced, insulin-treated cells. FIG. 9C Hepatic triglyceride content in rapamycin-treated Fc or N1-IC-transduced mice, sacrificed after a 16-h fast followed by 6-h refeeding. FIG. 9D Gene expression in rapamycin-treated Fc or N1-IC-transduced mice, sacrificed after a 16-h fast followed by 6-h refeeding. FIG. 9E: Glucose tolerance test in mice transduced with Fc or N1-IC, injected daily with rapamycin or vehicle. *p<0.05, **p<0.01 and ***p<0.001 vs. Cre− mice. FIG. 9F: AUC from GTT in mice transduced with Fc or N1-IC, injected daily with rapamycin or vehicle. AUC was normalized to Fc-transduced mice for each treatment. Nice were 10-week-old, short-term (3 weeks) HFD-fed C57B1/6 males. *p<0.05, **p<0.01 and ***p<0.001 vs. Cre− mice.

FIG. 10A-FIG. 10B: mTor inhibition prevents Notch-induced lipogenic gene expression. ***p<0.001 vs. Fc control, &p<0.001 vs. scrambled shRNA, #p<0.001 vs. no insulin, ̂p<0.001 vs. insulin, *p<0.001 vs. N1-IC+insulin. Data show means±SEM. FIG. 10A: Fasn-luciferase activity in FAO hepatoma cells transfected with scrambled (scr) or a second raptor shRNA (raptor2), transduced with Fc or N1-IC and treated with 100 nM insulin, or FIG. 10B: Fasn-luciferase activity in FAO hepatoma cells transfected with scrambled (scr) or a second raptor shRNA (raptor2), infected with Fc or N1-IC and treated with 100 nM insulin and/or 25 nM rapamycin.

FIG. 11A-FIG. 11H: Notch induces mTorc1 complex stability. FIG. 11A: Western blots of liver proteins from 5 h-fasted L-Rbpj and control mice (mTor and Actin blots reproduced from FIG. 2n). FIG. 11B: Western blot of liver protein from chow-fed, 12-week-old mice transduced with Fc or N1-IC, sacrificed at day 7, after overnight fasting. FIG. 11C: Western blots from FAO hepatoma cells transduced with either Fc or N1-IC, with or without treatment with MG132 for 4-h. FIG. 11D: Western blots of primary hepatocytes, transfected with Raptor cDNA, then transduced with Fc or Notch1 and treated for 2-h with cycloheximide. FIG. 11E: Fasn-luciferase activity in FAO hepatoma cells transduced with Fc or N1-IC, and co-transduced with GFP or Raptor. ***p<0.001 vs. Fc, &p<0.001 vs. N1-IC plus GFP. FIG. 11F: Western blots of HEK 293 cells transfected with Raptor-FLAG, followed by transduction with GFP or N1-IC and immunoprecipitation with anti-FLAG antibody. FIG. 11G Western blots of primary hepatocytes transfected with Raptor-FLAG, followed by transduction with GFP or N1-IC and immunoprecipitation with anti-FLAG antibody. Protein expression normalized to either actin or tubulin. Data show means ≡SEM. FIG. 11H: Model of Notch effects on hepatic glucose and lipid metabolism.

FIG. 12A-FIG. 12D: Notch induces mTorc1 complex stability. FIG. 12A: Endogenous Raptor gene expression in primary hepatocytes transduced with Fc or N1-IC, then treated with either vehicle (no tx) or insulin (ins) for 2-h. FIG. 12B: Western blots of primary hepatocytes, transfected with Raptor cDNA, then transduced with control (Fc) or Notch1 and treated for 2-h with insulin. FIG. 12C: Western blots from HEK 293 cells transfected with mTor-Myc, followed by transduction with GFP or N1-IC and immunoprecipitation with anti-Myc antibody in the presence of increasing concentrations of CHAPS (FIG. 12D). Data show means±SEM.

FIG. 13A-FIG. 13C: Hepatocyte specific ablation of Notch signaling protects from obesity-induced glucose tolerance and fatty liver. Despite unchanged body weight (FIG. 131), HFD-fed L-Rbpj mice show (FIG. 13B) improved glucose tolerance and (FIG. 13C) decreased hepatic steatosis.

FIG. 14A-FIG. 14D: Reduced Srebp1c-induced lipogenesis in L-Rbpj mice. *p<0.05, **p<0.01 and ***p<0.001 as compared to relevant control mice. FIG. 14A: HFD-fed Cre− and L-Rbpj mice were sacrificed after an overnight fast followed by 6 hours of refeeding prior to liver protein extraction and Western blot for lipogenic proteins. FIG. 14B De novo lipogenesis in hepatocytes from chow-fed 16-week-old mice (triplicate wells, representative of 2 individual experiments). FIG. 14C: Basal and insulin-stimulated Srebp1c expression in primary hepatocytes from chow-fed, 16-week-old Cre− and L-Rbpj littermates, transferred to serum-free medium for 16-h, followed by addition of 10 nM insulin for 6-h prior to lysis. FIG. 14D: Fasn-luciferase activity in primary hepatocytes from chow-fed, 16-week-old Cre− and L-Rbpj littermates, transferred to serum-free medium for 16-h, followed by addition of 10 nM insulin for 6-h prior to lysis.

FIG. 15A-FIG. 15B: Inhibition of Notch signaling increases Akt signaling, but reduces mTorc1 activity. FIG. 15A: L-Rbpj mice show higher Akt phosphorylation at the PDK1 site (T308) but lower mTorc1 activity, as assessed by p70 S6Kinase T389 or 4E-BP1 T37/46 phosphorylation. FIG. 15B: Hepatocytes derived from L-Rbpj mice show lower, whereas Cre− hepatocytes infected with N1-IC adenovirus higher mTorc1 activity.

FIG. 16A-FIG. 16D: Noth activity in liver increases with NASH. FIG. 16A: Liver Notch target gene (HES1) expression in patients underging percutaneous liver biopsy in evaluation of abnormal liver function tests, as compared to plasma ALT levels; or (FIG. 16B) NAS score (p<0.001 for both comparisons). FIG. 16C: Notch activity is highest in patients with NAFLD Activity score (NAS)>2. FIG. 16D: Hepatocyte Notch activity is increased in T2D and NASH as assessed by IF staining of Heyl and HeyL protein (red) levels. (blue—DAPI counterstain) *p<0.05, **p<0.01 and ***p<0.001 as comparted to NAS 0-2.

FIG. 17A-FIG. 17D: Notch activity in liver increases with MCDD or HFHC feeding. FIG. 17A: Liver Notch target gene expression mice fed MCDD or control diet for 2 weeks or (FIG. 17B) for up to 8 weeks. FIG. 17C: Liver Notch target gene expression mice fed HFHC or control diet for 16 weeks. FIG. 17D: ALT levels in chow (square) or HFHC (diamond)-fed mice is positively correlated with hepatic Heyl. *p<0.05, **p<0.01 and ***p<0.001 as comparted to control diet.

FIG. 18A-FIG. 18F: Notch Decoy protects from obesity-induced glucose intolerance and fatty liver. FIG. 18A: Hepatocyte Notch antagonism with Decoy adenoviral transduction improves glucose tolerance and (FIG. 18B) reduces hepatic triglyceride content in HFD-fed mice, independent of change in (FIG. 18C) body weight or (FIG. 18D)—adiposity. FIG. 18E: Notch Decoy increases insulin sensitivity (increased pAkt) while reducing mTorc1 signaling (reduced p-S6K), resulting in (FIG. 18F) less Srebp1c cleavage and resultant lower Fasn/Acc1 gene product expression. *p<0.05 as compared to Fc control.

FIG. 19A-FIG. 19D: Hepatocyte Jag1 expression increases with obesity. *p<0.05, **p<0.01 and ***p<0.001 as compared to NT; ND—not detected. FIG. 19A: Livers derived from leptin-deficient obese (ob/ob) mice and wildtype (NT) littermates sacrificed in the ad libitum state, prior to gene expression analysis of canonical Notch target genes. FIG. 19B: primary hepatocytes derived from leptin-deficient obese (ob/ob) mice and wildtype (NT) littermates sacrificed in the ad libitum state, prior to gene expression analysis of canonical Notch target genes. FIG. 19C: Notch ligand expression in livers from overnight fasted NT mice. FIG. 19D: Notch ligand expression in livers from obese, non-diabetic patients undergoing liver biopsy at time of bariatric surgery.

FIG. 20A-FIG. 20E: Notch decoy variants block ligand-specific Notch signaling. *p<0.05, **p<0.01 and ***p<0.001 as compared to no treatment or Fc control. FIG. 20A Notch decoy variants schematic. FIG. 20B: HEK 293 cells transiently transfected with Notch1 and a Notch reporter construct (CSL×3-luciferase) were co-cultured with Hela cells transfected with Notch ligand Jagged1; co-cultured cells were treated with gamma-secretase inhibitor (GSI), negative control (Fc) or parent (N1d1-24) and Notch decoy variants (N1d1-13 or N1d10-24). FIG. 20C: HEK 293 cells transiently transfected with Notch1 and a Notch reporter construct (CSL×3-luciferase) were co-cultured with Hela cells transfected with Notch ligand Delta-like 1; co-cultured cells were treated with gamma-secretase inhibitor (GSI), negative control (Fc) or parent (N1d1-24) and Notch decoy variants (N1d1-13 or N1d10-24). FIG. 20D: Hepa1clc7 hepatoma cells transfected with CSL×3-luciferase, then exposed to Fc, parent Notch decoy (1-24) or the two experimental decoy variants (1-13 or 10-24) produced and secreted by HEK 293 cells. FIG. 20E Fc control, N1d1-13 or N1d10-24 was expressed in liver of highfat diet fed mice by adenoviral transduction, liver RNA isolated and subjected to cDNA synthesis and quantitative PCR for Glucose-6-phosphatase (G6pc) and Sterol response element binding protein (Srebp1c) expression.

### DETAILED DESCRIPTION OF THE INVENTION

### Terms

As used in this application, except as otherwise expressly provided herein, each of the following terms shall have the meaning set forth below.

“Administering” may be effected or performed using any of the methods known to one skilled in the art. The methods comprise, for example, intralesional, intramuscular, subcutaneous, intravenous, intraperitoneal, liposome-mediated, transmucosal, intestinal, topical, nasal, oral, anal, ocular or otic means of delivery.

As used herein, the term “composition”, as in pharmaceutical composition, is intended to encompass a product comprising the active ingredient(s) and the inert ingredient(s) that make up the carrier, as well as any product which results, directly or indirectly from combination, complexation, or aggregation of any two or more of the ingredients, or from dissociation of one or more of the ingredients, or from other types of reactions or interactions of one or more of the ingredients.

As used herein, “effective amount” refers to an amount which is capable of treating a subject having a tumor, a disease or a disorder. Accordingly, the effective amount will vary with the subject being treated, as well as the condition to be treated. A person of ordinary skill in the art can perform routine titration experiments to determine such sufficient amount. The effective amount of a compound will vary depending on the subject and upon the particular route of administration used. Based upon the compound, the amount can be delivered continuously, such as by continuous pump, or at periodic intervals (for example, on one or more separate occasions). Desired time intervals of multiple amounts of a particular compound can be determined without undue experimentation by one skilled in the art. In one embodiment, the effective amount is between about 1 μg/kg-10 mg/kg. In another embodiment, the effective amount is between about 10 μg/kg-1 mg/kg. In a further embodiment, the effective amount is 100 μg/kg.

“Extracellular domain” as used in connection with Notch receptor protein means all or a portion of Notch which (i) exists extracellularly (i.e. exists neither as a transmembrane portion or an intracellular portion) and (ii) binds to extracellular ligands to which intact Notch receptor protein binds. The extracellular domain of Notch may optionally include a signal peptide (“sp”). “Extracellular domain”, “ECD” and “Ectodomain” are synonymous.

“Notch”, “Notch protein”, and “Notch receptor protein” are synonymous. In addition, the terms “Notch-based fusion protein” and “Notch decoy” are synonymous. The following Notch amino acid sequences are known and hereby incorporated by reference: Notch1 (Genbank accession no. S18188 (rat)); Notch2 (Genbank accession no. NP_{—}077334 (rat)); Notch3 (Genbank accession no. Q61982 (mouse)); and Notch4 (Genbank accession no. T09059 (mouse)). The following Notch nucleic acid sequences are known and hereby incorporated by reference: Notch1 (Genbank accession no. XM_{—}342392 (rat) and NM_{—}017617 (human)); Notch2 (Genbank accession no. NM_{—}024358 (rat), M99437 (human and AF308601 (human)); Notch3 (Genbank accession no. NM_{—}008716 (mouse) and XM_{—}009303 (human)); and Notch4 (Genbank accession no. NM_{—}010929 (mouse) and NM_{—}004557 (human)).

“Notch decoy protein”, as used herein, means a fusion protein comprising a portion of a Notch receptor protein which lacks intracellular signaling components and acts as a Notch signaling antagonist. Notch decoy proteins comprise all or a portion of a Notch extracellular domain including all or a portion of the EGF-like repeats present in the Notch extracellular domain. Examples of Notch decoy proteins include fusion proteins which comprise (a) amino acids, the sequence of which is identical to the sequence of a portion of the extracellular domain of a human Notch receptor protein and (b) amino acids, the sequence of which is identical to the sequence of an Fc portion of an antibody. In some Notch decoy proteins (b) is located to the carboxy terminal side of (a). Some Notch decoy proteins further comprise a linker sequence between (a) and (b). Notch decoy proteins can be selected from the group consisting of human Notch1 receptor protein, human Notch2 receptor protein, human Notch3 receptor protein and human Notch4 receptor protein. In some Notch decoy proteins the extracellular domain of the human Notch receptor protein is selected from the group consisting of Notch1 EGF-like repeats 1-36, Notch1 EGF-Like repeats 1-13, Notch1 EGF-like repeats 1-24, Notch1 EGF-like repeats 9-23, Notch1 EGF-like repeats 10-24, Notch1 EGF-like repeats 9-36, Notch1 EGF-like repeats 10-36, Notch1 EGF-like repeats 14-36, Notch1 EGF-like repeats 13-24, Notch1 EGF-like repeats 14-24, Notch1 EGF-like repeats 25-36, Notch4 EGF-like repeats 1-29, Notch4 EGF-like repeats 1-13, Notch4 EGF-like repeats 1-23, Notch4 EGF-like repeats 9-23, Notch4 EGF-like repeats 9-29, Notch4 EGF-Like repeats 13-23, and Notch4 EGF-like repeats 21-29.

Examples of Notch decoy proteins can be found in U.S. Pat. No. 7,662,919 B2, issued Feb. 16, 2010, U.S. Patent Application Publication No. US 2010-0273990 A1, U.S. Patent Application Publication No. US 2011-0008342 A1, U.S. Patent Application Publication No. US 2011-0223183 A1 AND PCT International Application No. PCT/US2012/058662; the entire contents of each of which are hereby incorporated by reference into this application.

The terms “polypeptide,” “peptide” and “protein” are used interchangeably herein, and each means a polymer of amino acid residues. The amino acid residues can be naturally occurring or chemical analogues thereof. Polypeptides, peptides and proteins can also include modifications such as glycosylation, lipid attachment, sulfation, hydroxylation, and ADP-ribosylation.

As used herein, “pharmaceutically acceptable carrier” means that the carrier is compatible with the other ingredients of the formulation and is not deleterious to the recipient thereof, and encompasses any of the standard pharmaceutically accepted carriers. Such carriers include, for example, 0.01-0.1 M and preferably 0.05 N phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

“Subject” shall mean any organism including, without limitation, a mammal such as a mouse, a rat, a dog, a guinea pig, a ferret, a rabbit and a primate. In one embodiment, the subject is a human.

“Treating” means either slowing, stopping or reversing the progression of a disease or disorder. As used herein, “treating” also means the amelioration of symptoms associated with the disease or disorder. Diseases include, but are not limited to, Tumor Angiogenesis, Atherosclerosis, Wound Healing, Retinopathy of Prematurity, Pre-eclampsia, Diabetic retinopathy, Ischemia, Stroke, Cardiovascular Disease, Psoriasis, lymphedema, tumorigenesis and tumor lymphangiogenesis, age-related macular degeneration (AND), wet AND, pancreatic cancer and breast cancer.

As used herein, an “agents for the treatment of fatty liver disease” are any agent known to or thought to treat a fatty liver disease. Agents for the treatment of obesity include, but are not limited to vitamin E, selenium, betadine, metformin, rosiglitazone, pioqlitazone, insulin sensitizers, antioxidants, probiotics, Omega-3 DHA, pentoxifylline, anti-TNF-alpha, FXR agonists and GLP-1 agonists.

Units, prefixes and symbols may be denoted in their SI accepted form. Unless otherwise indicated, nucleic acid sequences are written left to right in 5′ to 3′orientation and amino acid sequences are written left to right in amino- to carboxy-terminal orientation. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

### EMBODIMENTS OF THE INVENTION

The present invention provides a method of treating a subject suffering from a fatty liver disease which comprises administering to the subject a Notch decoy protein in an amount effective to treat the subject's fatty liver disease.

In one or more embodiments the fatty liver disease is alcoholic fatty liver disease.

In one or more embodiments the fatty liver disease is non-alcoholic fatty liver disease.

In one or more embodiments the fatty liver disease is non-alcoholic steatohepatitis.

In one or more embodiments the subject is also suffering from metabolic syndrome.

In one or more embodiments the subject is also suffering from diabetes.

In one or more embodiments the subject is also suffering from hypertension.

In one or more embodiments the subject is also suffering from obesity.

In one or more embodiments the subject is also suffering from dyslipidemia.

In one or more embodiments the Notch decoy protein comprises (a) amino acids, the sequence of which is identical to the sequence of a portion of the extracellular domain of a human Notch receptor protein and (b) amino acids, the sequence of which is identical to the sequence of an Fc portion of an antibody.

In one or more embodiments the human Notch receptor protein is selected from the group consisting of human Notch1 receptor protein, human Notch2 receptor protein, human Notch3 receptor protein and human Notch4 receptor protein.

In one or more embodiments the human Notch receptor protein is human Notch1 receptor protein.

In one or more embodiments the human Notch receptor protein is human Notch2 receptor protein.

In one or more embodiments the human Notch receptor protein is human Notch3 receptor protein.

In one or more embodiments the human Notch receptor protein is human Notch4 receptor protein.

In one or more embodiments the Fc portion of the antibody is the Fc portion of a human antibody.

In one or more embodiments (b) is located to the carboxy terminal side of (a).

In one or more embodiments the Notch decoy protein further comprises a linker sequence between (a) and (b).

In one or more embodiments the portion of the extracellular domain of the human Notch receptor protein is selected from the group consisting of Notch1 EGF-like repeats 1-36, Notch1 EGF-like repeats 1-13, Notch1 EGF-like repeats 1-24, Notch1 EGF-like repeats 9-23, Notch EGF-like repeats 10-24, Notch1 EGF-like repeats 9-36, Notch1 EGF-like repeats 10-36, Notch1 EGF-like repeats 14-36, Notch1 EGF-like repeats 13-24, Notch1 EGF-like repeats 14-24, Notch1 EGF-like repeats 25-36, Notch4 EGF-like repeats 1-29, Notch4 EGF-like repeats 1-13, Notch4 EGF-like repeats 1-23, Notch4 EGF-like repeats 9-23, Notch4 EGF-like repeats 9-29, Notch4 EGF-like repeats 13-23, and Notch4 EGF-like repeats 21-29.

In one or more embodiments the portion of the extracellular domain of the human Notch receptor protein is Notch1 EGF-like repeats 1-24.

In one or more embodiments the portion of the extracellular domain of the human Notch receptor protein is Notch1 EGF-like repeats 10-24.

In one or more embodiments the portion of the extracellular domain of the human Notch receptor protein is Notch1 EGF-like repeats 1-36.

In one or more embodiments, the Notch decoy protein inhibits Jagged-induced signalling.

In one or more embodiments, the Notch decoy protein only inhibits Jagged-induced signalling.

In one or more embodiments treating comprises reducing hepatic triglycerides.

In one or more embodiments the Notch decoy protein is administered in connection with a diet regimen.

In one or more embodiments the Notch decoy protein is administered in connection with an exercise regimen.

In one or more embodiments the Notch decoy protein is administered as a monotherapy.

In one or more embodiments the Notch decoy protein is administered in combination with one or more additional agents for the treatment of the fatty liver disease.

In one or more embodiments the one or more additional agents for the treatment of the fatty liver disease are selected from the group consisting of vitamin E, selenium, betadine, metformin, rosiglitazone, pioglitazone, insulin sensitizers, antioxidants, probiotics, Omega-3 DHA, pentoxifylline, anti-TNF-alpha, FXR agonists and GLP-1 agonists.

The present invention provides a composition comprising a pharmaceutically acceptable carrier and an amount of a Notch decoy protein effective to treat a fatty liver disease.

The present invention provides a package comprising:
(a) the pharmaceutical composition of the invention; and
(b) instructions for using the pharmaceutical composition of step (a) to treat the fatty liver disease.

The present invention also provides a method of treating a subject suffering from a fatty liver disease which comprises administering to the subject a Jagged inhibitor in an amount effective to treat the subject's fatty liver disease.

For the foregoing embodiments, each embodiment disclosed herein is contemplated as being applicable to each of the other disclosed embodiments. Thus, all combinations of the various elements described herein are within the scope of the invention.

### EXPERIMENTAL DETAILS

### First Series of Experiments

### Materials and Methods for First Series of Experiments

### Antibodies

We purchased antibodies to phospho-Akt1 (#2965), phospho-p70 S6K (#9205), total p70 S6k (#9202), phospho-mTor (#5536), total mTor (#2983), phospho-4E-BP1 (#2855), total 4E-BP1 (#9644), raptor (#2280), rictor (#2114), Gβ1 (#3274), fatty acid synthase (#3189), acetyl-CoA carboxylase (#3676), tubulin (#2148), and actin (#8456) from Cell Signaling, FLAG M2 (F1804) and c-Myc (C3956) from Sigma, anti-Srebp1c (NB600-582) from Novus, and anti-Val1744-cleaved Notch1 (ab52301) from Abcam.

### In Vivo Inhibitor Studies

Dibenzazepine (Syncom, 2 μmol/kg body weight), a γ-secretase inhibitor (GSI), and rapamycin (Enzo, 2 mg/kg body weight), were suspended in vehicle—0.5% Nethocel E4M (w/v, Colorcon) and 0.1% Tween-80 (Sigma) solution—and sonicated for two minutes to achieve a homogeneous suspension prior to daily (×5 days) intraperitoneal injection (van Es 2005).

### Experimental Animals

We crossed Albumin-cre (Postic 2000), Rbpj lox (Fujikura 2006) and Foxo1flox (Paik 2007) mice on C57BL/6 background to generate albumin(cre):Rbp-Jxflox/flox (L-Rbpj), albumin(cre):Foxo1flox/flox (L-Foxo1) and albumin(cre):Rbp-Jxflox/flox Foxo1flox/flox (L-Rbpj/Foxo1) mice; genotyping primers were previously described (Pajvani 2011) and only male mice were studied. Mice were weaned to either standard chow (Purina Mills #5053) or high-fat diet (Harlan Laboratories TD.06414). Wildtype C57Bl/6 (strain 0662) and leptin-deficient ob/ob (strain 0632) male mice were purchased from Jackson Labs. The Columbia University IACUC has approved all animal procedures.

### Metabolic Analyses

Blood glucose was measured by glucose meter (OneTouch) and plasma insulin by ELISA (Millipore). We performed glucose tolerance tests after a 16-hr (6 PM-10 AM) fast using intraperitoneal injection of 2 g/kg body weight glucose. Hepatic lipids were extracted (Folch 1957), normalized by either liver weight or protein, and confirmed by Oil Red O staining of snap-frozen liver sections. We used colorimetric assays to measure triglyceride (Thermo), cholesterol E (Wako) and non-esterified fatty acid (Wako). Hepatic de novo lipogenesis was determined by measuring the amount of newly synthesized FA, as resolved by TLC, present in the liver 1 h after intraperitoneal injection of 1 mCi of 3H2O (Zhang 2006). Triglyceride secretion rate was measured after injection of Poloxamer 407, with serial measurement of plasma triglycerides (Li 2011).

### Hepatocyte Studies

We isolated and cultured primary mouse hepatocytes as described (Pajvani 2011). For gene and protein expression studies, we pre-treated hepatocytes with 50 nM rapamycin (Cell Signaling) or vehicle for 30 min, followed by 6 h with 10 nM insulin (Sigma). We measured fatty acid oxidation as described (Li 2011), with the following modifications: primary hepatocytes were incubated serum-free medium with 1.5% fatty-acid free BSA (Sigma) containing 0.1 mM cold oleic acid and 1 μCi 14C-oleic acid (PerkinElmer Life Sciences) for 4 h. Labeled medium was transferred to flasks; 200 μl of 70% perchloric acid was injected into the bottom of the flask, 100 μL of N1 KOH was injected onto filter paper held by a center well, and the flasks were incubated for an additional 1 hour. Trapped 14CO2 on the alkalinized filter paper was measured as described (Li 2011). We measured lipogenesis as described (Yecies 2011), with the following modifications: hepatocytes were stimulated with 10 nM insulin in serum-free DNEM for 2 h, then labeled with 14C-acetate (PerkinElmer Life Sciences) for 2 h. After incubation with 3:2 hexane:isopropanol for 3 h, extracted lipids were dried under N2 gas, then resuspended in 2:1 chloroform:methanol prior to separation of lipid species by TLC and counting of labeled triglycerides. Counts were normalized to total cellular protein. All primary hepatocyte experiments were finished within 36 h after plating.

### Quantitative RT-PCR

We isolated RNA with Trizol (Invitrogen) or RNeasy mini-kit (Qiagen), synthesized cDNA with Superscript III RT (Invitrogen), and performed qPCR with a DNA Engine Opticon 2 System (Bio-Rad) and DyNAmo HS SYBR green (New England Biolabs). mRNA levels were normalized to 18s using the ΔΔC(t) method and are presented as relative transcript levels (Kitamura 2007).

### Adenovirus Studies

Notch1-IC, Notch decoy (1-24), Fc and GFP adenoviruses have been described (Kitamura 2007; Funahashi 2011; Nakae 2003). We transduced primary hepatocytes or HEK 293 cells at MOI 5 and FAO hepatoma cells at MOI 200, to achieve 90-100% infection efficiency as assessed by GFP expression. For in vivo studies, we injected 1×109 purified viral particles (Viraquest)/g body weight via orbital sinus; we performed metabolic analysis on days 3-5 and sacrificed the animals at day 7 or 14 post-injection. We limited analysis to mice showing 2-5-fold hepatic Notch1 overexpression or detectable hepatic Notch decoy or Fc expression by Western blot.

### Luciferase Assays

We transfected (Lipofectamine 2000, Invitrogen) FAO hepatoma cells or primary hepatocytes with a luciferase construct (Addgene 18890) containing the proximal (−220 to +25) Fasn promoter sequence (Kim 1998). In some experiments, we co-transfected plasmids containing shRNA to Raptor (Addgene #21339 or #21340) or Rictor (Addgene #21341), with scrambled shRNA (Addgene #1864) as a control (Peterson 2009), and/or transduced cells with Notch1-IC or control (Fc) adenovirus. 24 h after transfection, FAO cells were transferred to serum-free medium with or without 100 nM insulin (Sigma) for 16 h prior to lysis and luciferase measurements as described (Kim 1998).

### Immunoprecipitations

HER 293, FAG, and primary hepatocytes were lysed in 0.3% or 0.6% CHAPS-containing buffer (Kim 2002), followed by immunoprecipitation for 2-h at 4° C., and overnight elution prior to Western blot analysis (Qiang 2012).

### Statistical Analysis

We used two-way ANOVA to analyze the data. All Westerns were quantitated using NIH lmageJ software. Data represent means±SEM.

### Results

Hepatic Notch Action Peaks Twice, after Prolonged Fasting and at Late Refeeding

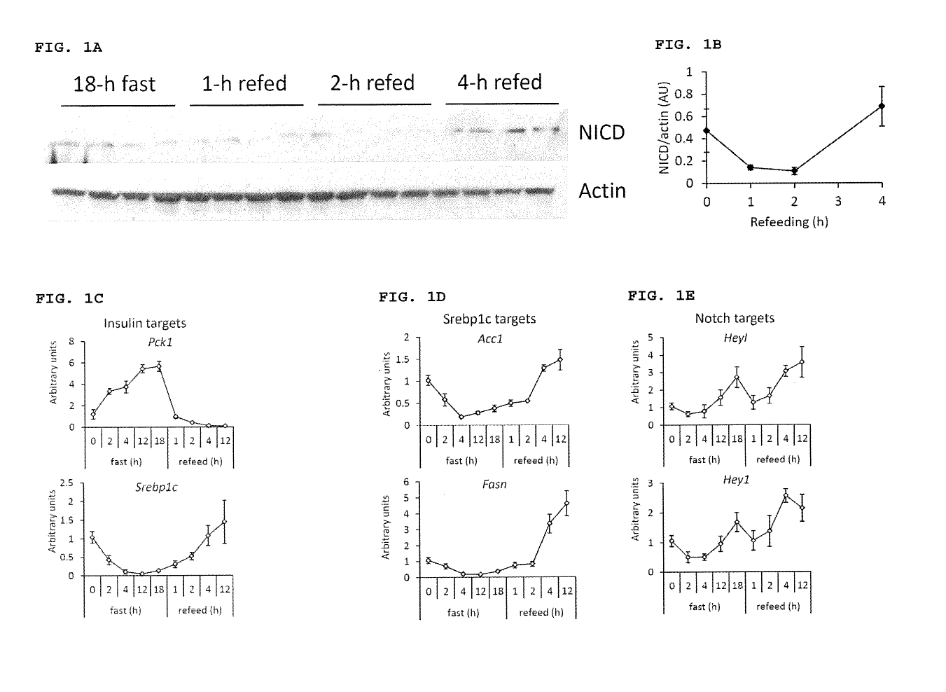

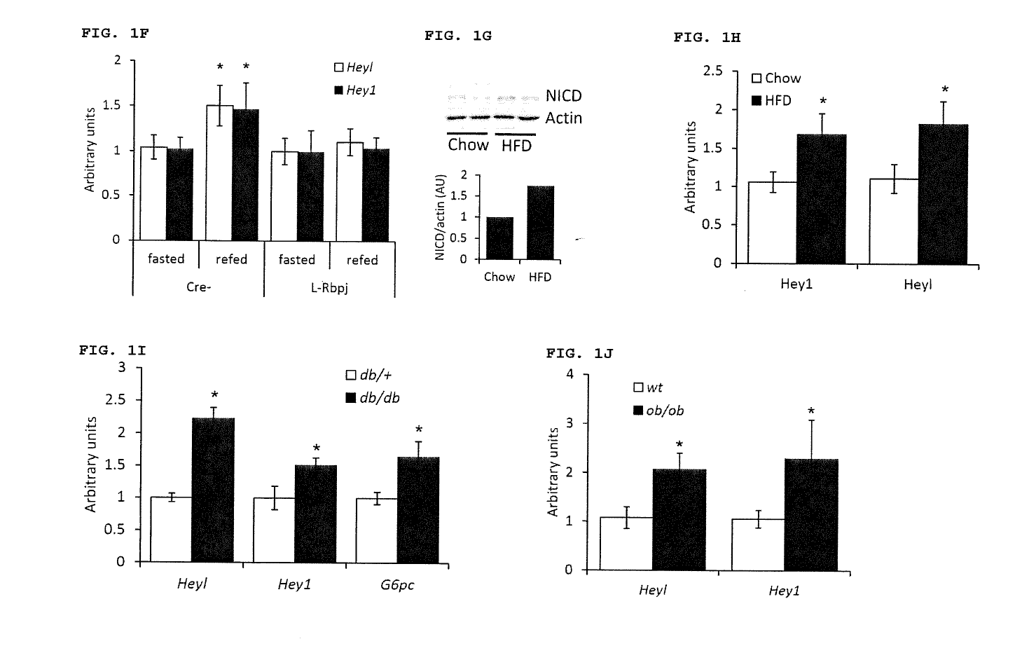

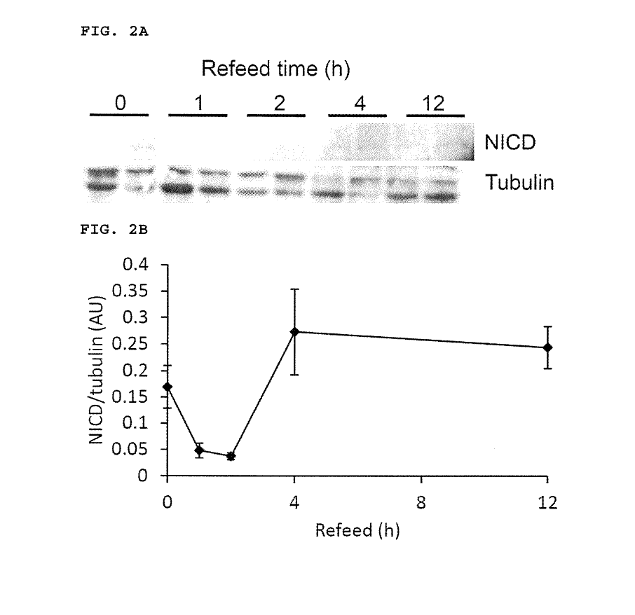

Notch1 activation in liver, as reflected by cleavage at Val1744 and increased expression of Notch targets, increased with fasting (Pajvani 2011). In early refeeding (0-2 h), Notch cleavage and target gene expression declined, followed by a second peak of Notch activation at later time points (4-12 h) (FIG. 1A, FIG. 1B and FIG. 2A, FIG. 2B). Notably, Notch activation during fasting coincides with increased gluconeogenic gene expression, while the second peak coincides with expression of Srebp1c and its targets (Fatty acid synthase, Fasn; and Acetyl-CoA-carboxylase, Acc1) (FIG. 1C-FIG. 1E), as well as activation of mTor (not shown). This induction was expectedly absent in livers from mice lacking hepatocyte Rbp-Jk (L-Rbpj) (FIG. 1F) (Pajvani 2011), confirming that classical Notch activation is affected by the nutritional state. Livers from mice fed a high-fat diet (HFD) also showed greater Notch activation than chow-fed littermates (FIG. 1G, FIG. 1H), as did hepatocytes and livers from leptin-signaling deficient mice (FIG. 1I, FIG. 1J), suggesting a cell-autonomous dysregulation of Notch signaling in obesity and fatty liver.

Liver-Specific Deletion of Rbp-Jk Protects from Diet-Induced Steatosis

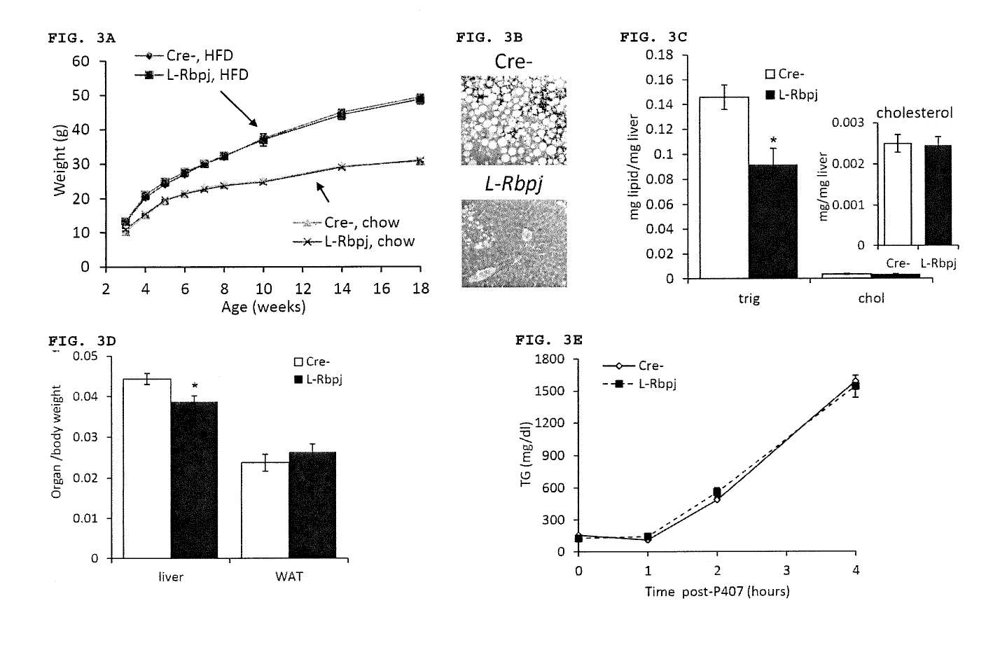

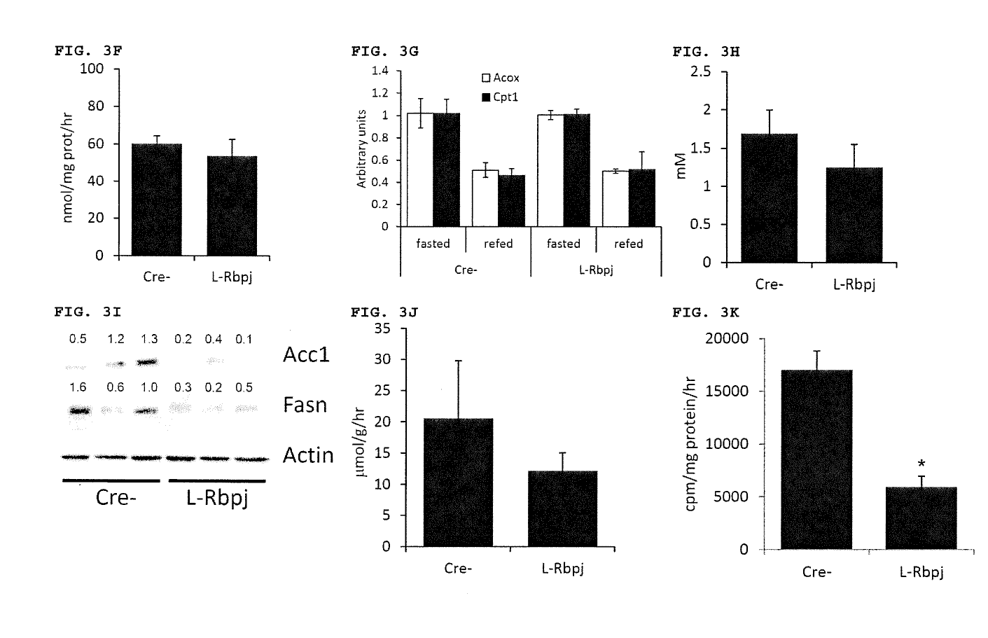

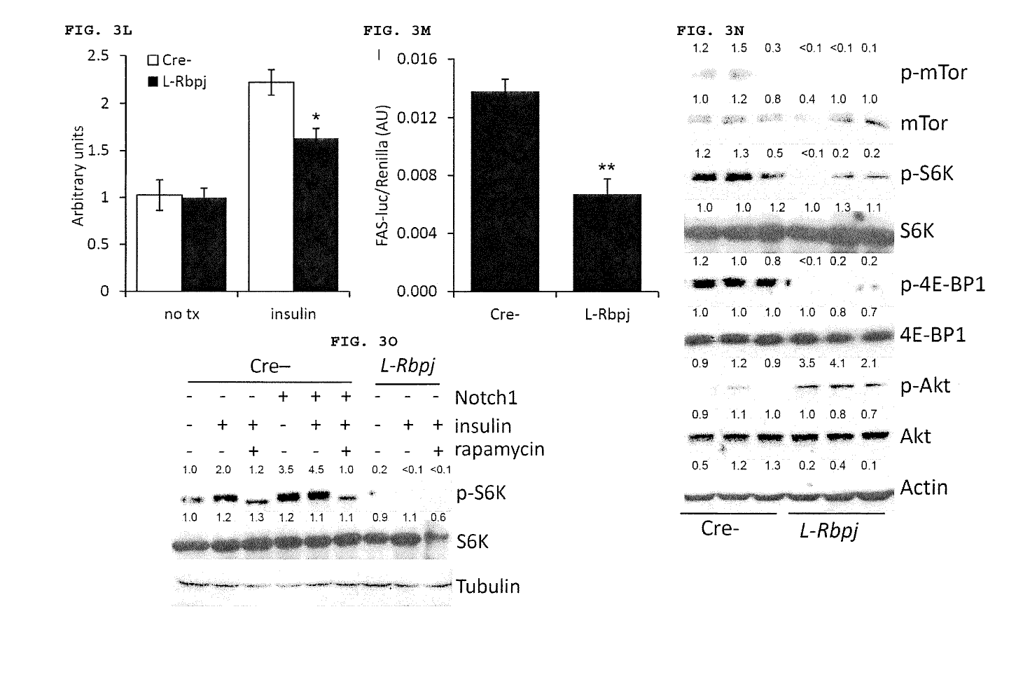

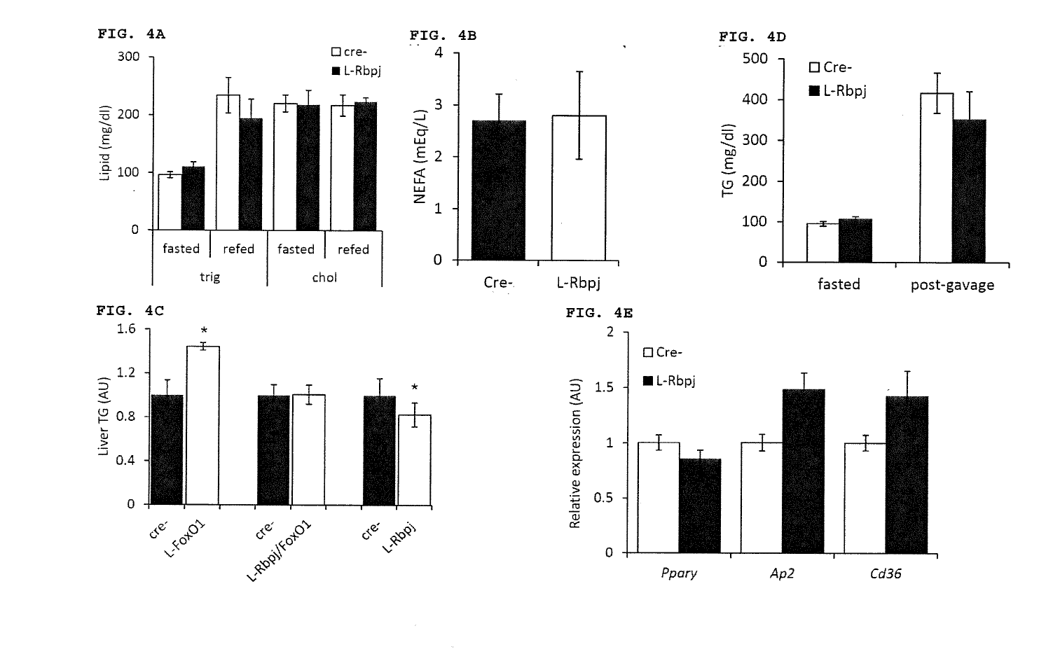

As whole-body disruption of Rbp-Jk results in embryonic lethality,16 we generated liver-specific Rbp-Jk knockout (L-Rbpj) mice, in which hepatocyte Rbp-Jk was deleted post-natally,20 with full recombination by 6-12 weeks of age.23 We have previously shown that chow- or HFD-fed L-Rbpj mice are protected from insulin resistance (Pajvani 2011). Given the interaction between Pbp-Jk and FoxO1 (Kitamura 2007), we hypothesized that L-Rbpj mice would have similarly increased hepatic triglyceride as mice lacking liver FoxOs (Haeusleer 2012; Tao 2011). L-Rbpj mice showed normal body weight under different diets (FIG. 3A), but markedly lower HFD-induced hepatic steatosis, due to a 30-50% reduction in hepatic triglycerides without effects on hepatic cholesterol levels (FIG. 3C, FIG. 3D). L-Rbpj mice showed reduced liver weight without changes in adiposity (FIG. 3D) or serum lipids (FIG. 4A, FIG. 4B). Reduced hepatic triglyceride content was also seen in chow-fed or short-term (3 week) HFD-fed L-Rbpj mice. Moreover, Rbp-Jk knockout prevented steatosis in mice lacking hepatic FoxO1 (FIG. 4C) (Haeusler 2012), suggesting that the Notch pathway regulates hepatic lipid deposition independent of FoxO1.

### L-Rbpj Mice Show Reduced De Novo Lipogenesis

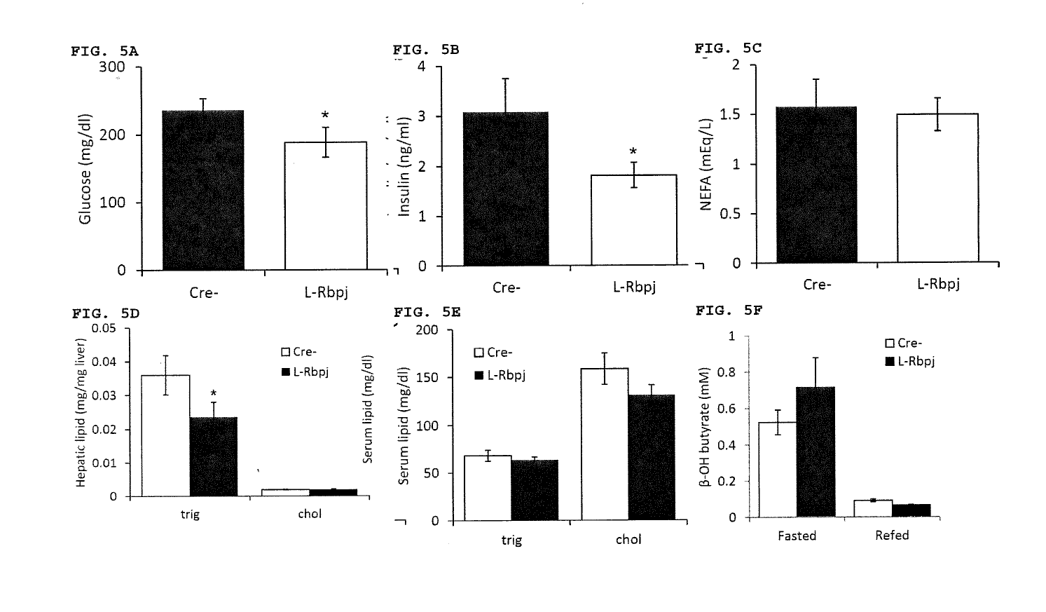

We evaluated cell-autonomous and non-autonomous pathways that regulate hepatic triglyceride accumulation (Savage 2010; Postic 2008). VLDL secretion was unaltered in L-Rbpj mice (FIG. 3E), as were liver expression of fatty acid oxidation enzymes Acox and Cpt1a, serum ketones, β-oxidation of exogenous fatty acids in primary hepatocytes (FIG. 3F-FIG. 3H), and plasma triglyceride levels after olive oil gavage (FIG. 4D). Next, we studied lipogenesis—L-Rbpj livers showed reduced Fasn and Acc1 expression (FIG. 3I), and a trend towards reduced fatty acid production after injection of tritiated water (FIG. 3J). In Rbp-Jk-deficient primary hepatocytes, we found significantly repressed 14C-acetate incorporation into triglyceride (FIG. 3K), reduced insulin-dependent Srebp1c expression (FIG. 3L), and reduced expression of a luciferase reporter construct driven by the proximal Fasn promoter containing a consensus Srebp1c binding site (Kim 1998) (FIG. 3M). Alternative lipogenic pathways, including PPARγ signaling, were unaltered in L-Rbpj mice (FIG. 4e) (Zhang 2006). These data indicate that blocking hepatic Notch results in reduced hepatic triglyceride, likely due to impaired lipogenesis. We observed a similar protection from insulin resistance associated with reduced hepatic lipid content following short-term HFD (FIG. 5A-FIG. 5F).

Reduced mTorc1 Signaling in L-Rbpj Mice

We studied the main signaling pathways implicated in lipogenesis, insulin/Akt and nutrient/mTor.3 As we reported, insulin signaling was increased in L-Rbpj liver, with increased Akt phosphorylation at the Pdk1 site, T308 (Pajvani 2011. Conversely, we noted a marked reduction of hepatic mTorc1 signaling, as indicated by decreased phosphorylation of mTor and mTorc1 targets, p70 S6 kinase and 4E-BP1 (FIG. 3N) (Gingras 1999; Chiang 2005; Weng 1998). To determine if this effect was cell-autonomous, we isolated primary hepatocytes from Cre- and L-Rbpj mice, and found that Akt phosphorylation was higher (data not shown), while basal and insulin-stimulated p70 S6k phosphorylation were lower (FIG. 3O). These data suggest that Notch-dependent transcriptional activity is required for hepatocyte mTorc1 activity.

Acute Notch Inhibition Protects from Diet-Induced Insulin Resistance and Fatty Liver

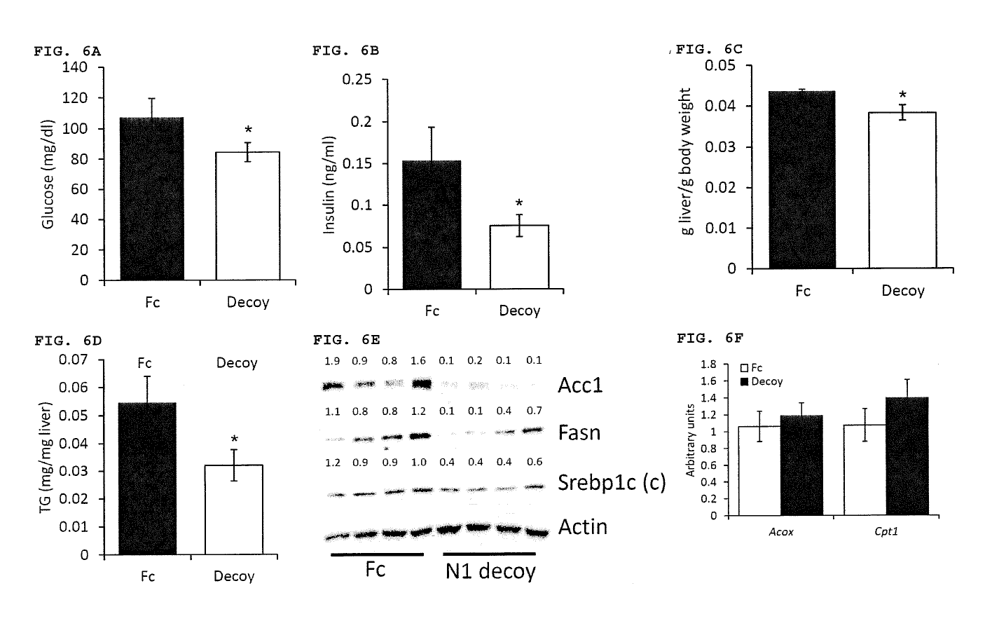

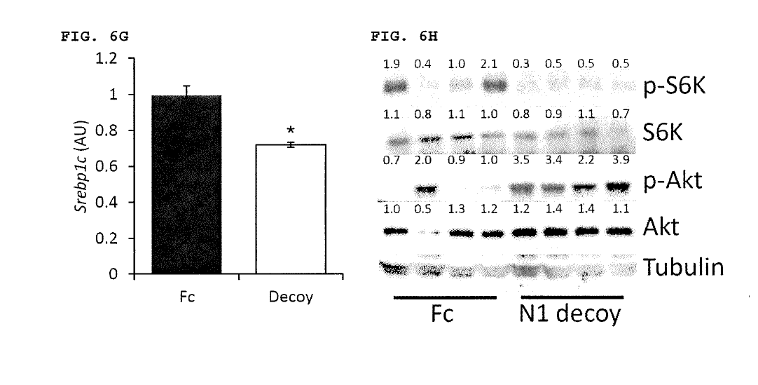

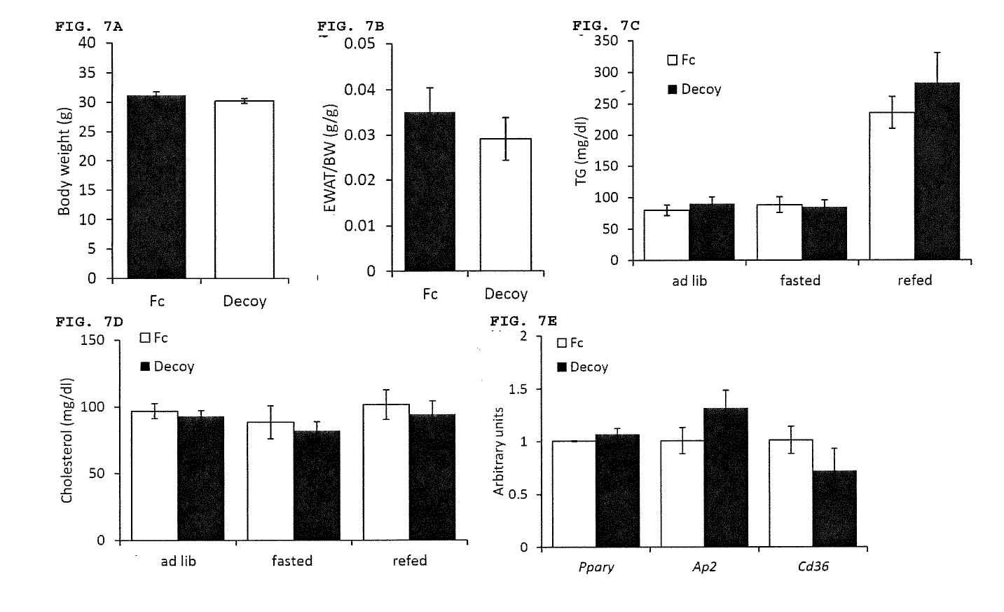

Given these surprising findings, and to exclude the possibility of a developmental phenotype in L-Rbpj mice, we tested if acute inhibition of Notch signaling can similarly protect from diet-induced fatty liver and reduce mTorc1 function. We transduced adult mice with a “decoy” Notch1 receptor that encodes only the extracellular domain (Funahashi 2008; Funahashi 2011) and acts in a dominant-negative manner by sequestering endogenous ligand. Adenovirus-driven Notch1 decoy is preferentially expressed in the liver, and is poorly secreted into the circulation (data not shown). Consistent with results from L-Rbpj mice, Notch decoy administration to HFD-fed mice lowered glucose and insulin levels (FIG. 6A, FIG. 6B), and reduced liver weight and triglyceride content (FIG. 6C, FIG. 6D), without affecting body or adipose weight (FIG. 7A, FIG. 7B). Notch decoy reduced Srebp1c cleavage, and Fasn and Acc1 expression (FIG. 6E). We observed no difference in fatty acid oxidation genes (FIG. 6F) or serum lipids (FIG. 7C, FIG. 7D). We transduced primary hepatocytes with Notch decoy and observed reduced Srebp1c expression (FIG. 6G), but no change in Pparγ or its targets (FIG. 7E), suggesting that acute inhibition of hepatocyte Notch reduces Srebp1c-directed lipogenesis in a cell-autonomous manner. Similar to L-Rbpj mice, livers from Notch decoy-transduced mice demonstrated increased pAkt-T308, but lower pS6k-S389 (FIG. 6H). These data indicate that acute reduction in Notch signaling increases insulin sensitivity, while lowering mTorc1 and hepatic triglyceride content.

Hepatic Overexpression of Notch1 Induces mTorc1 Signaling and Fatty Liver

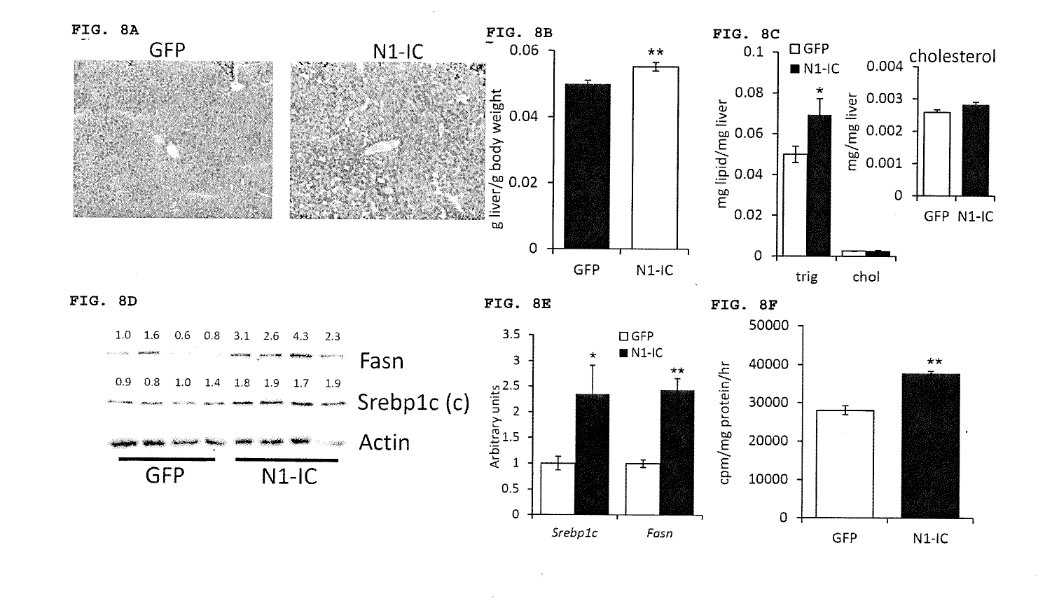

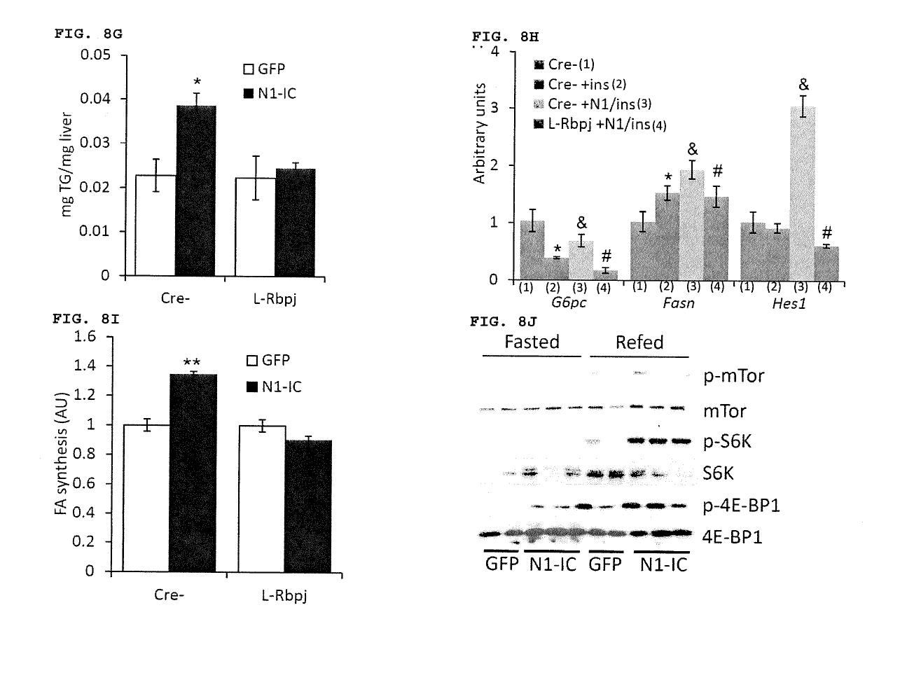

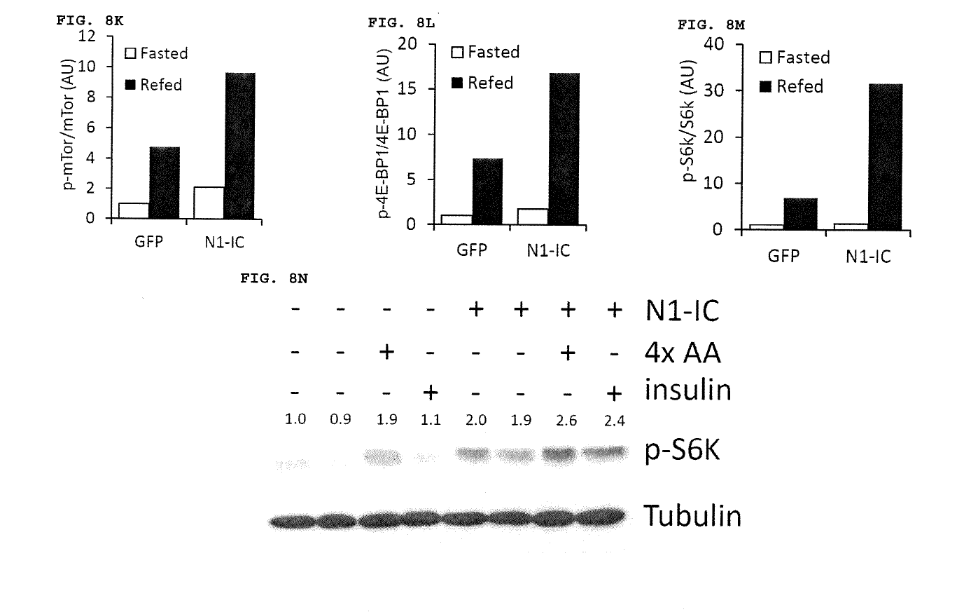

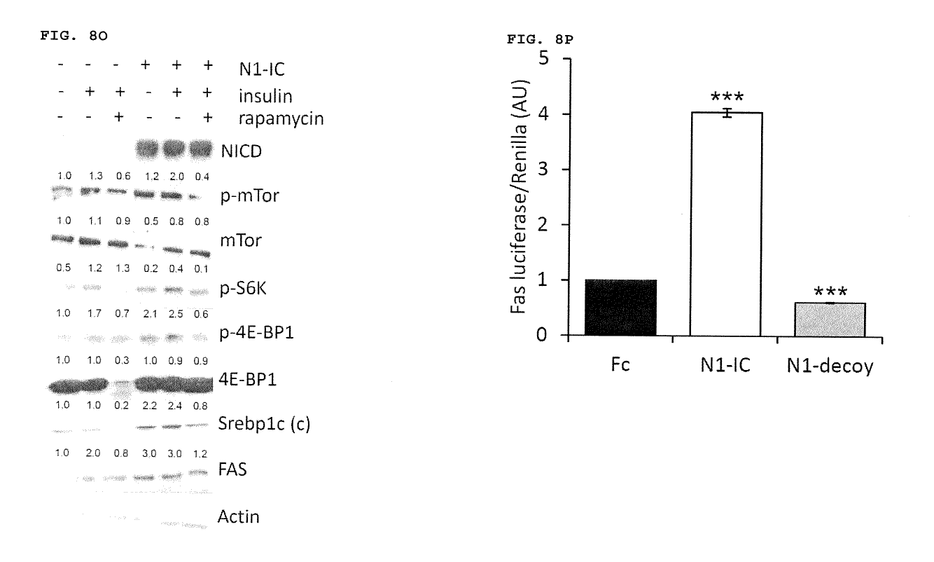

Our loss-of-function studies suggest that Notch signaling is permissive for mTorc1 activation and diet-induced steatosis. We thus tested whether Notch gain-of-function would be sufficient to increase mTorc1 function and induce fatty liver in vivo. Chow-fed mice transduced with adenovirus encoding constitutively active Notch1 (N1-IC) showed a ˜40% increase in hepatic triglyceride and increased liver weight (FIG. 8A-FIG. 8C), without concomitant changes in body weight or composition (data not shown). N1-IC-transduced livers demonstrated higher Srebp1c cleavage, and increased expression of Srebp1c and Fasn (FIG. 8D, FIG. 8E). Consequently, primary hepatocytes transduced with N1-IC showed greater lipogenesis (FIG. 8F). Importantly, N1-IC expression failed to increase hepatic lipid, gene expression and fatty acid synthesis in L-Rbpj mice and hepatocytes (FIG. 8G-I), suggesting that Notch-induced lipogenesis requires Rbp-Jk, similar its activation of hepatic glucose production (Pajvani 2011).

The increase of lipogenic genes induced by N1-IC was paralleled by increased hepatic mTorc1 activity in fasted and (more markedly) refed animals (FIG. 8J-FIG. 8M), consistent with enhanced physiologic regulation of mTor activity. Similarly, activation of mTorc1 signaling by insulin and amino acids was potentiated by N1-IC (FIG. 8N), suggesting that Notch modulates but does not override endogenous mTor regulation. To confirm that induction in mTorc1 signaling and lipogenic gene expression is cell-autonomous, we transduced primary hepatocytes with N1-IC, and detected increased mTor signaling, greater Srebp1c cleavage and higher levels of Fasn protein and mRNA (FIG. 8O). In hepatoma cells, Notch activity correlated with Fasn-luciferase reporter activation, again consistent with a cell-autonomous effect (FIG. 8P).

Inhibition of mTor Prevents Notch-Induced Lipogenic Gene Expression and Fatty Liver

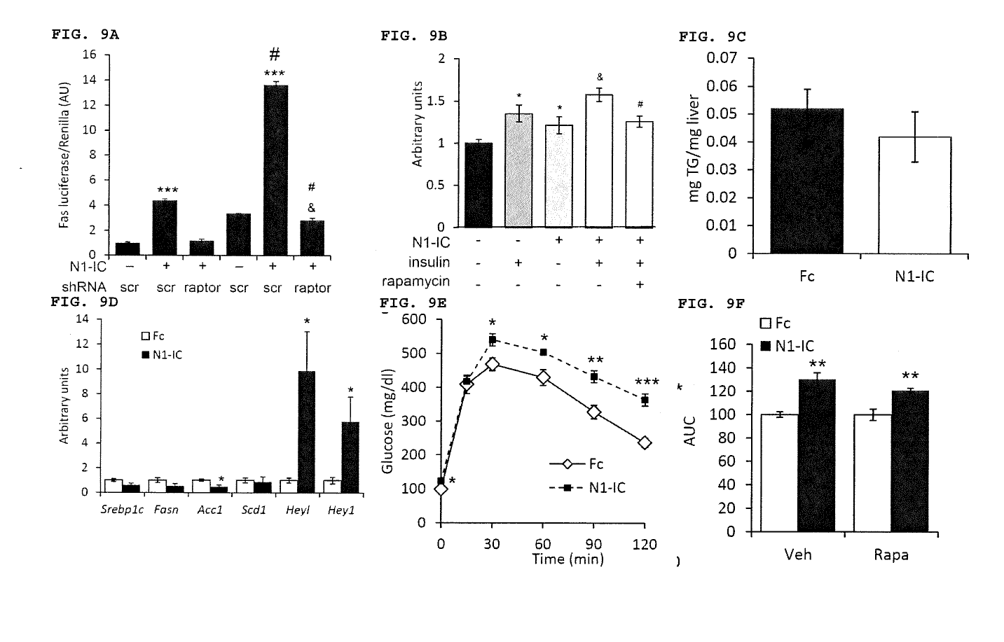

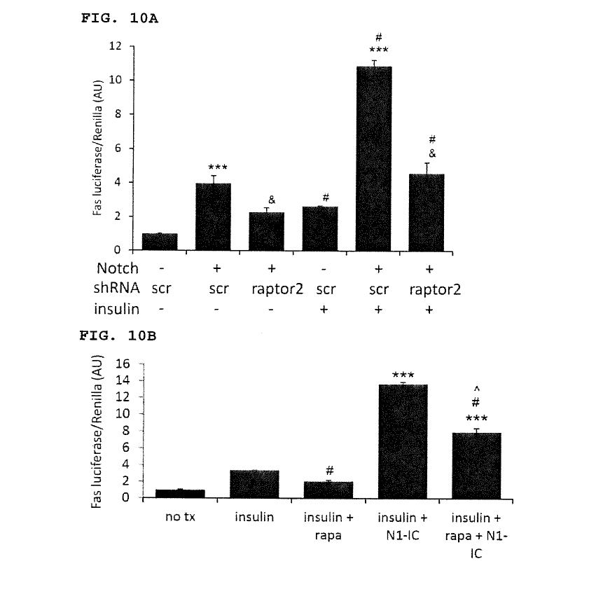

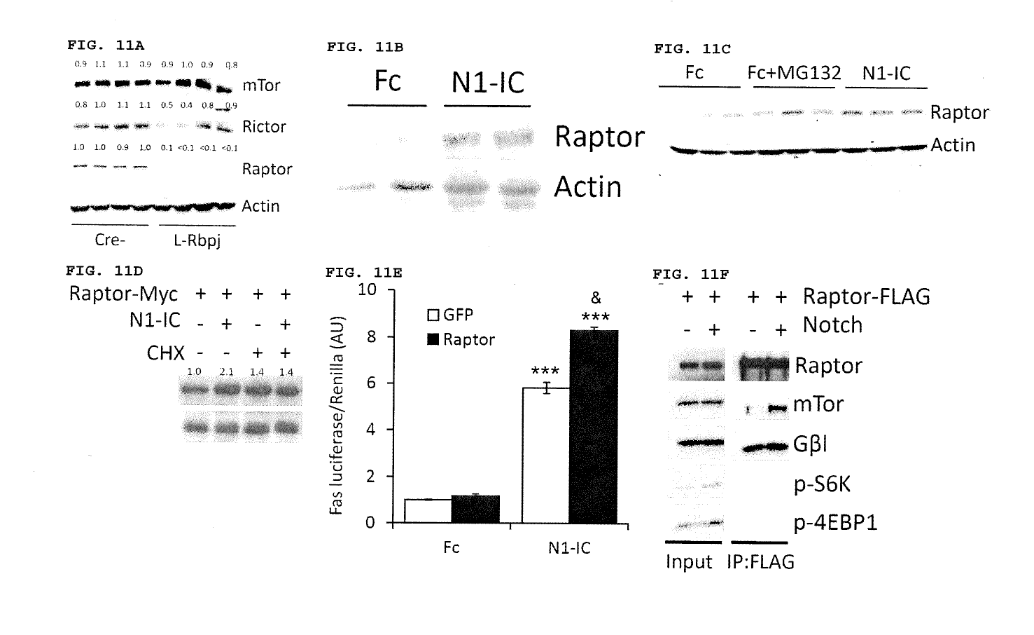

To test the hypothesis that Notch induction of lipogenic gene expression and fatty liver requires mTorc1 signaling, we co-transfected hepatoma cells with Fasn-luciferase and shRNA to Raptor (Peterson 2009), the defining component of the mTorc1 complex, then transduced cells with N1-IC adenovirus. N1-IC promoted basal as well as insulin-stimulated Fasn-luciferase activity; Raptor shRNA reversed both effects, which was potentiated by insulin, but reversed by Raptor knockdown (FIG. 9A). We saw similar results with a second shRNA to Raptor, as well as with rapamycin treatment (FIG. 10A, FIG. 10B), suggesting that N1-IC-induced Fasn expression is mTorc1-dependent. Similarly, Notch-induction of endogenous Fasn in primary hepatocytes was augmented by insulin, and suppressed by rapamycin (FIG. 9B), confirming that Notch activates lipogenesis through mTorc1, and not through secondary effects on insulin signaling.

Based on these data, we hypothesized that the increase in lipogenic gene expression and fatty liver seen in mice transduced with N1-IC adenovirus would be ameliorated by rapamycin treatment. Indeed, N1-IC increased hepatic triglyceride and lipogenic gene expression in vehicle-treated mice, while these effects were completely reversed by rapamycin treatment (FIG. 9C, FIG. 9D, as compared to FIG. 8D, FIG. 8E). The effect of rapamycin was specific to Notch induction of lipogenic genes, as Heyl and Heyl were unaffected (FIG. 9D). Similarly, although rapamycin induced mild glucose intolerance (data not shown) (Blattler 2012), N1-IC-transduced mice showed further exacerbation (FIG. 9E, FIG. 9F). These data show that Notch induction of hepatic steatosis, but not its induction of glucose intolerance, can be reversed by rapamycin treatment.

Notch Increases mTorc1 Complex Stability

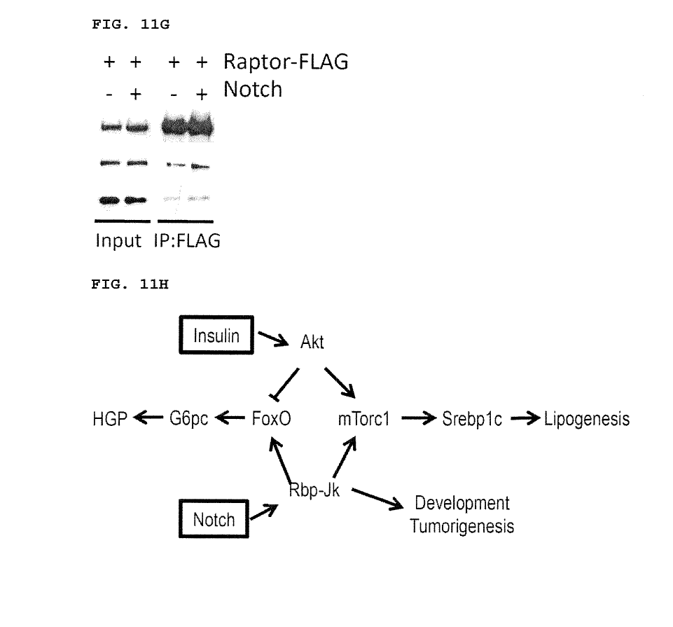

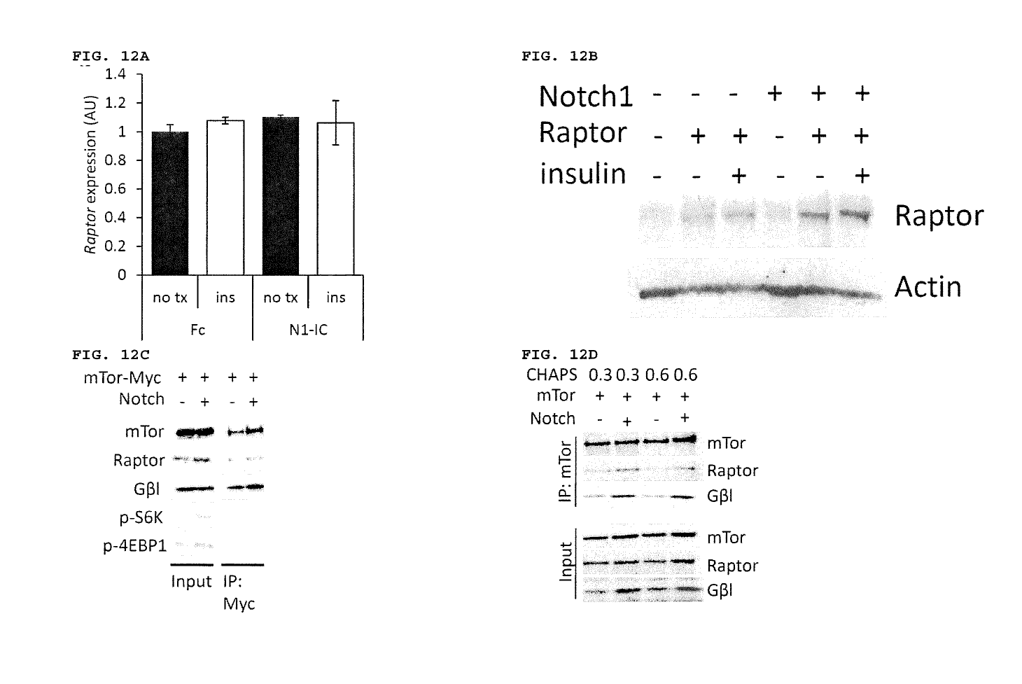

To study the mechanism of altered Notch-induced mTorc1 activation, we examined mTor complex levels in L-Rbpj mouse liver. We found unchanged levels of the shared mTorc1/mTorc2 components, mTor and Gβ1, and of the mTorc2-specific component Rictor, but a surprising reduction in the levels of Raptor protein (FIG. 11A), independent of changes in Raptor mRNA (not shown), suggesting that the effects of Pbp-Jk deficiency are post-transcriptional. Conversely, mice transduced with N1-IC adenovirus demonstrated increased Raptor in liver (FIG. 11B). We found a similar increase of endogenous Raptor protein in hepatoma cells (FIG. 11C) or primary hepatocytes (not shown) transduced with N1-IC, without changes in Raptor mRNA (FIG. 12A). Transient transfection of Raptor cDNA in primary hepatocytes showed a similar effect, demonstrating that the action of Notch is independent of locus effects (FIG. 12B). Interestingly, the effect of N1-IC was independent of proteosomal inhibition by MG132 (FIG. 11C), but was fully reversed by treatment of hepatocytes with the protein synthesis inhibitor, cycloheximide (FIG. 11D).

Raptor overexpression did not suffice to induce Fasn-luciferase, consistent with previous work that Raptor overexpression per se does not increase mTorc1 function (Funahashi 2011; Peterson 2009), whereas co-expression of N1-IC and Raptor produced a synergistic effect (FIG. 11E). Likewise, overexpression of Raptor was insufficient to activate mTorc1 in either primary hepatocytes or HEK 293 cells (data not shown). We conclude that Notch induction of Raptor levels parallels, but does not cause increased mTorc1 activation, and hypothesized that increased Raptor levels are secondary to higher mTorc1 complex stability. Indeed, we found that Notch overexpression increased association among mTorc1 components in HEK 293 cells (FIG. 11F and FIG. 12C), and primary hepatocytes (FIG. 11G). Notch-stabilized mTorc1 complexes were resistant to increasing concentrations of CHAPS detergent known to disrupt the mTor-Raptor interaction (FIG. 12D)(Foster 2010; Kaizuka 2010; Kim 2002). These data indicate that the Notch stabilizes and activates mTorc1, resulting in increased de novo lipogenesis and fatty liver.

### Discussion for First Series of Experiments

The homeostatic functions of Notch in the adult animal have received less attention, except in neoplastic processes (Weinmaster 2006). We have shown that liver Notch signaling is regulated in response to metabolic stimuli, and that Notch1 increases hepatic glucose production by co-activating FoxO1 at the Glucose-6-phosphatase promoter (Pajvani 2011). Conversely, liver-specific deletion of Rbp-Jk (L-Rbpj mice), or t-secretase inhibitor (GSI) treatment improves glucose tolerance, and reduces hepatocyte glucose production.20 Interestingly, previous studies demonstrated that Notch1 can activate mTorc1 in leukemic cells, whereas GSIs decrease mTorc1 activity in breast cancer (Chan 2007; Efferson 2010). Thus, we hypothesized that hepatic Notch could modulate the coordinate actions of insulin on gluconeogenesis (via FoxO1) and lipogenesis (via mTorc1). We describe here that inhibition of hepatic Notch protects from obesity-induced fatty liver, likely through decreased de novo lipogenesis. Conversely, constitutive hepatic Notch signaling increases lipogenesis, fatty liver and activation of hepatic mTorc1 signaling, by stabilizing the mTorc1 complex. We show that Notch-mediated hepatic steatosis is rapamycin-sensitive, whereas Notch-induced glucose tolerance is mTor-independent. These results establish Notch as a unique pharmacological target in liver, whose inhibition can prevent the twin abnormalities of hepatic insulin resistance—excessive glucose production as well as fatty liver—by virtue of its ability to uncouple Akt from mTor.

The role of developmental pathways in metabolic homeostasis of adult tissues is only beginning to be appreciated (Liu 2011). We have shown that genetic or pharmacologic inhibition of Notch protects from diet-induced glucose intolerance, without effects on body weight or adiposity, in a FoxO1-dependent manner (Pajvani 2011). In this work, we demonstrate a similar protection from fatty liver with inhibition of hepatic Notch signaling. This is unexpected, as inhibition of hepatic FoxO1 is associated with increased hepatic lipid deposition (Haeusler 2012; Tao 2012; Matsumoto 2006; Haeusler 2010), an increasingly recognized effect of shifting hepatic carbon flux from glucose to lipid production (Sun 2012). In this regard, it appears that chronic (L-Rbpj mice) or acute Notch inhibition (Notch decoy), achieves the long-sought goal of decreasing hepatic glucose production without compensatory increases in hepatic lipid content. Interestingly, GSIs also induce fatty liver, but do so in a Notch-independent fashion (U.B.P., manuscript in preparation), consistent with the idea that substrates of γ-secretase include Notch-unrelated pathways, and restricting the repertoire of therapeutically viable Notch inhibitors that can be pursued for treatment of metabolic disease. Nonetheless, the many potential benefits of Notch inhibition, which include amelioration of atherosclerosis (Fukuda 2012), provide in our opinion a strong rationale to pursue Notch inhibition as a treatment of the metabolic syndrome (Kim-Muller 2011).

The identification of Notch as a regulator of carbon flux towards hepatic glucose or lipid production (FIG. 11I) is a conceptual advance, as is the surprising finding that a molecular pathway thought to be specialized toward differentiation is regulated by physiologic (fasting/re-feeding), as well as pathologic (insulin resistance) metabolic cues in hepatocytes. We hypothesize that in the overfed and insulin-resistant state, Notch signaling is inappropriately activated, and reprises its developmental interactions with FoxO1 and mTorc1. The mechanisms underlying nutritional activation of hepatic Notch require further clarification. For example, it should be determined whether Notch activation in the hepatocyte requires input from neighboring hepatocytes or other resident liver cells (endothelial, stellate, Kupffer, etc.). Similarly, which of the five Notch ligands drives signaling in response to nutrients is unknown, and the possibility that different ligands signal in different metabolic states to direct carbon flux or drive differentiation is teleologically attractive.

Besides the further validation of hepatic Notch as a therapeutic target, our data demonstrate a physiologic, and potentially pharmacologic, means of regulating mTorc1 activity and lipogenesis. Previous studies have indicated that tight control of hepatic mTorc1 signaling is critical for hepatic lipid metabolism (Peterson 2011; Yecies 2011). The tandem findings of mTorc1 stabilization and activation by Notch deserve further study. Since the identification of Raptor as the mTorc1-regulatory subunit, it has been known that the mTor-Raptor association is sensitive to detergent concentrations; 38 subsequent reports have confirmed this finding and identified potential post-translational modifications on Raptor (Foster 2010; Kaizuka 2010; Gwinn 2008) but none have been shown to mediate mTor-Raptor interaction. How Notch induces mTorc1 stability, and how precisely that translates to greater mTorc1 activation remain unclear. The demonstration that Raptor levels are decreased in LP-Rbpj mice and that cycloheximide prevents Notch-induced stabilization indicates that a transcriptional target(s) of Notch regulates complex stability.

In summary, Notch antagonism uncouples Akt from mTor activation, suggesting that Notch antagonists from oncology and neuroscience (Noguera-Troise 2006; Wu 2010) may be repurposed to treat fatty liver and diabetes. Furthermore, as Notch-mediated mTorc1 activation does not appear to be cell type-specific, modulators of mTorc1 processing and degradation may represent a therapeutic avenue to blockmTorc1 activity without the metabolic liabilities of current mTor inhibitors (Blattler 2012).

### REFERENCES FOR FIRST SERIES OF EXPERIMENTS

Bayard M et al. Am. Fam. Physician 73(11) June 1; 1961-8 (2006).
Blattler, S. N., et al. Yin Yang 1 deficiency in skeletal muscle protects against rapamycin-induced diabetic-like symptoms through activation of insulin/IGF signaling. Cell metabolism 15, 505-517 (2012).
Bolos, V., Grego-Bessa, J. & de la Pompa, J. L. Notch signaling in development and cancer. Endocrine reviews 28, 339-363 (2007).
Chan, S. M., Weng, A. P., Tibshirani, R., Aster, J. C. & Utz, P. J. Notch signals positively regulate activity of the mTOR pathway in T-cell acute lymphoblastic leukemia. Blood 110, 278-286 (2007).
Chiang, G. G. & Abraham, R. T. Phosphozylation of mammalian target of rapamycin (mTOR) at Ser-2448 is mediated by p70S6 kinase. The Journal of biological chemistry 280, 25485-25490 (2005).
Dufraine, J., Funahashi, Y. & Kitajewski, J. Notch signaling regulates tumor angiogenesis by diverse mechanisms. Oncogene 27, 5132-5137 (2008).
Efferson, C. L., et al. Downregulation of Notch pathway by a gamma-secretase inhibitor attenuates AKT/mammalian target of rapamycin signaling and glucose uptake in an ERBB2 transgenic breast cancer model. Cancer research 70, 2476-2484 (2010).
Folch, J., Lees, M. & Sloane Stanley, G. H. A simple method for the isolation and purification of total lipides from animal tissues. The Journal of biological chemistry 226, 497-509 (1957).
Fortini, N. E. Notch signaling: the core pathway and its posttranslational regulation. Developmental cell 16, 633-647 (2009).
Foster, K. G., et al. Regulation of mTOR complex 1 (mTORC1) by raptor Ser863 and multisite phosphorylation. The Journal of biological chemistry 283, 80-94 (2010).
Fujikura, J., et al. Notch/Rbp-j signaling prevents premature endocrine and duccal cell differentiation in the pancreas. Cell Metab 3, 59-65 (2006).
Fukuda, D., et al. Notch ligand Delta-like 4 blockade attenuates atherosclerosis and metabolic disorders. Proceedings of the National Academy of Sciences of the United States of America (2012).
Funahashi, Y., et al. A notch1 ectodomain construct inhibits endothelial notch signaling, tumor growth, and angiogenesis. Cancer research 68, 4727-4735 (2008).
Funahashi, Y., et al. Notch modulates VEGF action in endothelial cells by inducing Matrix Netalloprotease activity. Vascular cell 3, 2 (2011).
Gingras, A. C., et al. Regulation of 4E-BP1 phosphorylation: a novel two-step mechanism. Genes & development 13, 1422-1437 (1999).
Gwinn, D. N., et al. AMPK phosphorylation of raptor mediates a metabolic checkpoint. Molecular cell 30, 214-226 (2008).
Haeusler, R. A., Kaestner, K. H. & Accili, D. FoxOs function synergistically to promote glucose production. The Journal of biological chemistry 285, 35245-35248 (2010).
Haeusler, R. A., Pratt-Hyatt, N., Welch, C. L., Klaassen, C. D. & Accili, D. Impaired generation of 12-hydroxylated bile acids links hepatic insulin signaling with dyslipidemia. Cell metabolism 15, 65-74 (2012).
Hagiwara, A., et al. Hepatic mTORC2 Activates Glycolysis and Lipogenesis through Akt, Glucokinase, and SREBP1c. Cell metabolism 15, 725-738 (2012).
Houde, V. P., et al. Chronic rapamycin treatment causes glucose intolerance and hyperlipidemia by upregulating hepatic gluconeogenesis and impairing lipid deposition in adipose tissue. Diabetes 59, 1338-1348 (2010).
Howell, J. J. & Manning, B. D. mTOR couples cellular nutrient sensing to organismal metabolic homeostasis. Trends in endocrinology and metabolism: TEM 22, 94-102 (2011).
Kaizuka, T., et al. Tti1 and Tel2 are critical factors in mammalian target of rapamycin complex assembly. The Journal of biological chemistry 265, 20109-20116 (2010).
Kim, D. H., et al. mTOR interacts with raptor to form a nutrient-sensitive complex that signals to the cell growth machinery. Cell 110, 163-175 (2002).
Kim, J. B., et al. Nutritional and insulin regulation of fatty acid synthetase and leptin gene expression through ADD1/SREBP1. The Journal of clinical investigation 101, 1-9 (1998).
Kim, J. B., Wright, H. M., Wright, N. & Spiegelman, B. M. ADD1/SREBP1 activates PPARgamma through the production of endogenous ligand. Proceedings of the National Academy of Sciences of the United States of America 95, 4333-4337 (1998).
Kim-Muller, J. Y. & Accili, D. Cell biology. Selective insulin sensitizers. Science 331, 1529-1531 (2011).
Kitamra, T., et al. A Foxo/Notch pathway controls myogenic differentiation and fiber type specification. The Journal of clinical investigation 117, 2477-2485 (2007).
Lamming, D. W., et al. Rapamycin-induced insulin resistance is mediated by mTOPC2 loss and uncoupled from longevity. Science 335, 1638-1643 (2012).
Li, G., Hernandez-Ono, A., Crooke, P.n., Graham, M. J. & Ginsberg, H. N. Effects of antisense-mediated inhibition of 11beta-hydroxysteroid dehydrogenase type 1 on hepatic lipid metabolism. Journal of lipid research 52, 971-981 (2011).
Li, S., Brown, M. S. & Goldstein, J. L. Bifurcation of insulin signaling pathway in rat liver: mTORC1 required for stimulation of lipogenesis, but not inhibition of gluconeogenesis. Proceedings of the National Academy of Sciences of the United States of America 107, 3441-3446 (2010).
Liu, H., et al. Wnt signaling regulates hepatic metabolism. Science signaling 4, ra6 (2011).
Matsumoto, N., Han, S., Kitamura, T. & Accili, D. Dual role of transcription factor FoxO1 in controlling hepatic insulin sensitivity and lipid metabolism. The Journal of clinical investigation 110, 2464-2472 (2006).
Millar, J. S., Cromley, D. A., McCoy, N. G., Rader, D. J. & Billheimer, J. T. Determining hepatic triglyceride production in mice: comparison of poloxamer 407 with Triton WR-1339. Journal of lipid research 46, 2023-2028 (2005).
Nakae, J., et al. The forkhead transcription factor Foxo1 regulates adipocyte differentiation. Dev Cell 4, 119-129 (2003).
Noguera-Troise, I., et al. Blockade of D114 inhibits tumour growth by promoting non-productive angiogenesis. Nature 444, 1032-1037 (2006).
Oka, C., et al. Disruption of the mouse RBP-J kappa gene results in early embryonic death. Development 121, 3291-3301 (1995).
Paik, J. H., et al. FoxOs Are Lineage-Restricted Redundant Tumor Suppressors and Regulate Endothelial Cell Homeostasis. Cell 126, 309-323 (2007).
Pajvani, U. B., et al. Inhibition of Notch signaling ameliorates insulin resistance in a FoxO1-dependent manner. Nature medicine 17, 961-967 (2011).
Peterson, T. R., ec al. DEPTOR is an mTOR inhibitor frequently overexpressed in multiple myeloma cells and required for their survival. Cell 137, 873-886 (2009).
Peterson, T. A., et al. mTOR complex 1 regulates lipin 1 localization to control the SPEBP pathway. Cell 146, 408-420 (2011).
Postic, C. & Girard, J. Contribution of de novo fatty acid synthesis to hepatic steatosis and insulin resistance: lessons from genetically engineered mice. The Journal of clinical investigation 118, 829-838 (2008).
Poetic, C. & Magnuson, M. A. DNA excision in liver by an albumin-Cre transgene occurs progressively with age. Genesis 26, 149-150 (2000).
Qiang, L., ec al. Brown Remodeling of White Adipose Tissue by SirT1-Dependent Deacetylation of Ppargamma. Cell 150, 620-632 (2012).
Rizzo, P., et al. Rational targeting of Notch signaling in cancer. Oncogene 27, 5124-5131 (2008).
Sabatini, D. M. mTOR and cancer: insights into a complex relationship. Nature reviews. Cancer 6, 729-734 (2006).
Savage, D. B. & Semple, R. K. Recent insights into fatty liver, metabolic dyslipidaemia and their links to insulin resistance. Current opinion in lipidlogy 21, 329-336 (2010).
Shen, J., et al. Skeletal and CNS defects in Presenilin-1-deficient mice. Cell 89, 629-639 (1997).
Sun, Z., et al. Hepatic Hdac3 promotes gluconeogenesis by repressing lipid synthesis and sequestration. Nature medicine 18, 934-942 (2012).
Swiatek, P. J., Lindsell, C. E., del Amo, F. F., Weinmaster, G. & Gridley, T. Notch1 is essential for postimplantation development in mice. Genes & development 8, 707-719 (1994).
Tao, R., ec al. Hepatic FoxOs regulate lipid metabolism via modulation of expression of the nicotinamide phosphoribosyltransferase gene. The Journal of biological chemistry 268, 14681-14690 (2011).
van Es, J. H., et al. Notch/gamma-secretase inhibition turns proliferative cells in intestinal crypts and adenomas into goblet cells. Nature 435, 959-963 (2005).
Wang, Y. C., McPherson, K., Marsh, T., Goztmaker, S. L. & Brown, N. Health and economic burden of the projected obesity trends in the USA and the UK. Lancet 373, 815-825 (2011).
Weinmaster, G. & Kopan, R. A garden of Notch-ly delights. Development 133, 3277-3282 (2006).
Weng, Q. P., et al. Regulation of the p70 S6 kinase by phosphorylation in vivo. Analysis using site-specific anti-phosphopeptide antibodies. The Journal of biological chemistry 273, 16621-16629 (1998).
Wu, Y., et al. Therapeutic antibody targeting of individual Notch receptors. Nature 464, 1052-1057 (2010).
Yecies, J. L., et al. Akt stimulates hepatic SREBP1c and lipogenesis through parallel mTORC1-dependent and independent pathways. Cell metabolism 14, 21-32 (2011).
Zhang, Y. L., et al. Aberrant hepatic expression of PPARgamma2 stimulates hepatic lipogenesis in a mouse model of obesity, insulin resistance, dyslipidemia, and hepatic steatosis. The Journal of biological chemistry 211, 37603-37615 (2006).

### Second Series of Experiments

### T2D and NAFLD are Inadequately Treated with Currently Available Therapy

Obesity leads to insulin resistance, which begets the fasting hyperglycemia of Type 2 diabetes (T2D) (Lin 2011). In a parallel process, compensatory hyperinsulinemia drives hepatic de novo lipogenesis (Savage 2010), mediated in part by the nutrient-sensitive mechanistic target of rapamycin (mTOR) pathway (Li 2010). Increased lipogenesis, coupled with excess fatty acid flux to liver and impaired ability to catabolize and export these fatty acids (Bugianesi 2005), produces non-alcoholic fatty liver disease (NAFLD). NAFLD may be associated with hepatocellular damage and inflammation which predisposes to cirrhosis and hepatocellular carcinoma, but also further exacerbates hepatic insulin resistance through activation of FoxO1 (Rametta 2012), the key transcriptional activator of hepatic glucose production (Lin 2011). This vicious cycle results in coincident NAFLD and T2D, which show independent associations with cardiovascular disease and all-cause mortality (Villanova 2005).

There is no approved pharmacologic therapy for NAFLD, and although there are multiple T2D therapies available, none show durability and long-term efficacy (Kahn 2006). Novel pathways are sought to both further our understanding of the pathophysiology of insulin resistance as well as provide potential new pharmaceutical targets to assist in our management of obesity-related morbidity and mortality.

NAFLD May Lead to NASH, which has No Approved Pharmacotherapy.

NAFLD ranges in severity from simple steatosis, to hepatocellular damage and necroinflammatory changes which define non-alcoholic steatohepatitis (NASH). Regrettably, this progression of simple steatosis, a potential “pre-disease” state with prevalence approaching 30% in some populations (Bhala 2013), to NASH, which predisposes to cirrhosis and hepatocellular carcinoma, is unpredictable for any given patient (Loria 2010). In fact, the majority of patients diagnosed with excess hepatic fat will never develop NASH (Bhala 2013), which makes the lack of molecular signature of the NAFLD-NASH transition challenging to non-invasively track or develop drugs to block (Malik 2009; Hashimoto 2009). Recent evidence suggests a “two-hit” hypothesis (Day 1998), where the first hit of fat accumulation sensitizes the liver to further injury, mediated by cross-talk between hepatocytes and other liver residents to accelerate a fairly benign process to one that has severe clinical consequence without approved pharmacologic therapy (Hashimoto 2009; Carpino 2013). Available livers for transplantation will not keep pace with the expected growth in NASH incidence over the next few decade.

### Notch Bridges Two Nutrient-Sensitive Pathways in Hepatocytes—Insulin/FoxO1 and Nutrient/mTORC1.

In metabolically healthy liver, insulin represses glucose production, primarily by Akt-mediated phosphorylation and nuclear exclusion of FoxO1 (Matsumoto 2007), and promotes fatty acid synthesis from acetate (lipogenesis) by both transcriptional and non-transcriptional increase in Srebp1c activity (Shinomura 1999; Yecies 2011; Engelking 2004; Matsuda 2001). In the insulin-resistant state, however, FoxO1-mediated hepatic glucose production is unrestrained, resulting in hyperglycemia (Matsumoto 2006), but insulin stimulation of Srebp1c function is “paradoxically” increased, contributing to fatty liver (Liang 2002).

Notch is an evolutionarily conserved regulatory pathway of normal development, and is inappropriately re-activated in leukemia and other tumors (Bolos 2007). It is in these contexts that Notch has been shown to intersect with insulin and nutrient signaling pathways. FoxO1 physically interacts with the Notch transcriptional effector, Rbp-Jk, to co-regulate Notch-mediated differentiation processes (Kitamura 2007). In addition, Notch activates mTOR complex 1 (mTORC1) signaling in leukemic cells, and pharmacologic Notch inhibitors reduce mTOPC1-mediated oncogenic potential (Chan 2007; Efferson 2010). These observations provoked our hypothesis that Notch may similarly interact with the FoxO1 and mTORC1 signaling pathways in normal tissue, and may modulate the coordinate actions of insulin on hepatic glucose production (via FoxO1) and lipid synthesis (via mTOPC1).

The feasibility of this hypothesis depended on whether Notch signaling is present in developed liver—this was a legitimate question, as Notch is so critical to normal differentiation (Oka 1995; Swiatek 1994), that its potential role in fully differentiated tissue has not been adequately explored. Our initial characterization in murine liver demonstrated that the Notch pathway is active in healthy, adult mice and physiologically modulated by nutrient availability, but markedly increased in mouse models of obesity and insulin resistance (Pajvani 2011; Pajvani 2013). These results prompted a similar survey in liver biopsy specimens from patients—while Notch activity was evident in all patient liver samples, we observed increased Notch activation in patients with both T2D and NAFLD (Valenti et al.). Surprisingly, Notch activity showed independent and greatest correlation with NAFLD activity score (WAS) and plasma aminotransferase (ALT) levels, markers of the transition from NAFLD to NASH (Valenti et al.). In combination with our mouse studies, these data suggest that Notch may be a “druggable” common thread of the multiple hit model of NASH development (Yilmaz 2012), and that Notch inhibitors may block both obesity-induced lipid accumulation and the hepatocyte damage/inflamatory state that follows.

Inhibition of Hepatic Notch Signaling Leads to Weight-Independent improvements in Hepatic Glucose and Lipid Metabolism.

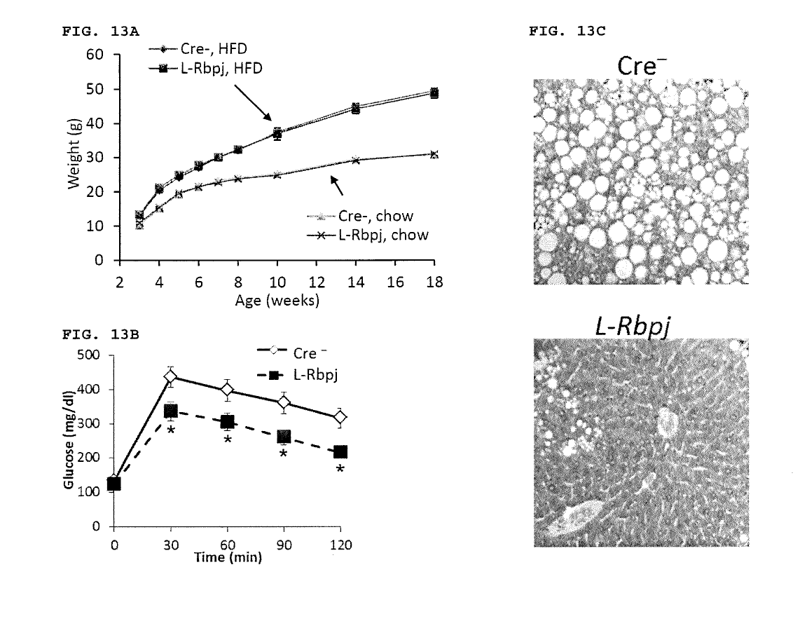

We next hypothesized that increased Notch activity is causative of, and not just correlated to, obesity-induced T2D and NAFLD. To investigate the repercussion of decreased Notch signaling, the most efficient means is to disrupt the common transcriptional effector of all 4 Notch receptors, Pbp-Jk (Oka 1995). As Pbp-Jk knockout animals show embryonic lethality (Oka 1995), we generated liver-specific Rbp-Jk knockout (Albumin-cre:Rbpj fl/fl mice, henceforth L-Rbpj) mice, which show gradual, post-natal recombination in hepatocytes (Pajvani 2011; Postic 2000). L-Rbpj mice showed no developmental defects, and normal liver histology, and gain weight at a comparable rate as control animals (FIG. 13A). As Rbp-Jk synergizes with FoxO1, the key transcriptional mediator of hepatocyte glucose production, to modulate differentiation (Kitamura 2007), we hypothesized that L-Rbpj mice would show reduced hepatic glucose production similar to liver-specific FoxO1 knockout (L-Foxo1) mice. As predicted, L-Rbpj mice are protected from high-fat diet (HFD)-induced glucose intolerance (FIG. 13B), which we established as cell-autonomous, by direct FoxO1-independent binding of Rbp-Jk to the Glucose-6-phosphatase (G6pc) promoter (Pajvani 2011).

L-Rbpj Mice Show Reduced Lipogenesis, Leading to Protection from Fatty Liver.

We predicted that L-Rbpj mice would have similarly increased hepatic triglyceride (TG) as mice lacking liver FoxOs (Haeusler 2012: Tao 2011). Unexpectedly, L-Rbpj mice were protected from HFD-induced hepatic steatosis (FIG. 13C). We hypothesized a cell-autonomous mechanism for the observed decrease in liver TG, but broadly evaluated potential causes, in L-Rbpj mice (Savage 2010; Postic 2008). In summary, we found:

### Cell Non-Autonomous:

Intestinal absorption of dietary, or exogenous (gavaged), lipids was unaltered.
Adipose lipolysis was unchanged, with unchanged Atg1 and Hs1 expression and normal free fatty acids.

### Cell-Autonomous:

VLDL secretion was unchanged, leading to normal plasma TG and similar response in Cre and L-Rbp) mice to lipoprotein lipase inhibition with Poloxamer 407 (Millar 2005).
Liver expression of fatty acid oxidation enzymes Acox and Cpt1a, serum ketones and β-oxidation of exogenous fatty acids in primary hepatocytes were unchanged.

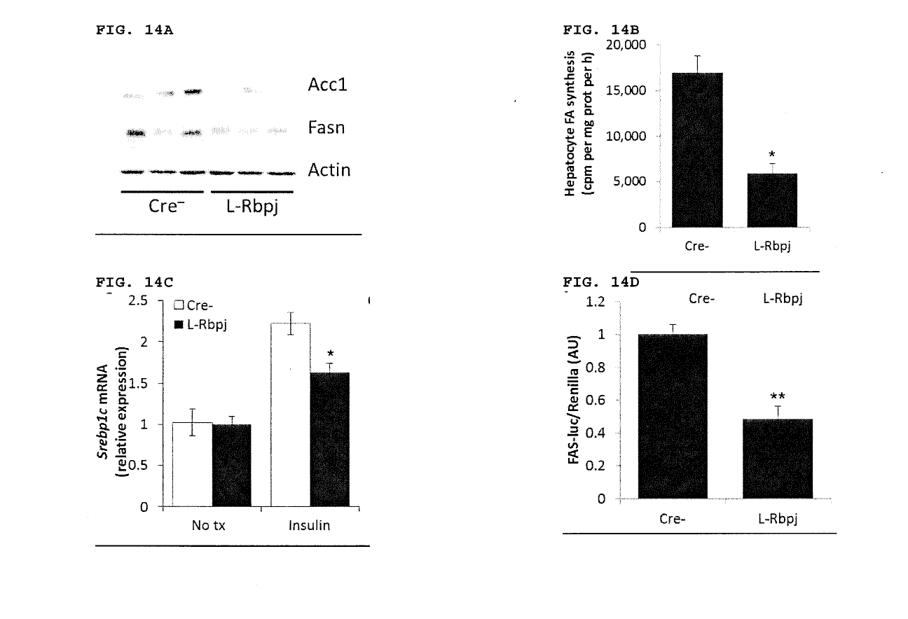

Next, we studied lipogenesis—first, we measured expression of lipogenic proteins, with focus on Fatty acid synthetase (Fasn) and Acetyl-CoA-Carboxylase (Acc1), rate-limiting enzymes in the manufacture of long-chain fatty acids from two-carbon precursors (Postic 2008). L-Rbpj mice express less liver Fasn and Acc1 (FIG. 14A); correspondingly, hepatocytes derived from L-Rbpj mice showed lower fatty acid synthesis (FIG. 14B). As Fasn and Acc1 are transcriptional targets of the insulin and nutrient-activated, lipogenic transcription factor Srebp1c (Shinomura 1999; Liang 2002; Horton 2002), we hypothesized that L-Rbpj mice have decreased Srebp1c activity. Indeed, we found Impaired insulin-dependent Srebp1c expression, and activity, as assessed by lower expression of Fasn promoter-driven luciferase containing a consensus Srebp1c binding site (Kin 1998) (FIG. 14C, FIG. 14D).

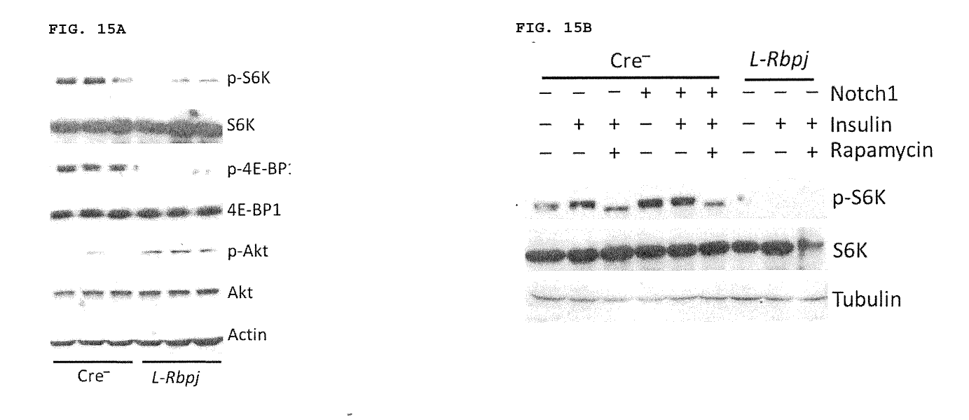

These data suggest that lower hepatic TG in L-Rbpj mice is due to impaired Srebp1c-mediated lipogenesis. We next studied pathways that converge on Srebp1c—insulin/Akt and nutrient/mTOR (Li 2010). The mTOR protein kinase functions in two multi-protein complexes which have multiple common (mTOR, Gβ1, deptor) and several unique components, most notably Raptor for mTORC1, and Rictor for mTORC2 (Sabatini 2006). Activation of the nutrient-sensing mTORC1 pathway stimulates hepatic de novo lipogenesis (Li 2010), regulating insulin and Srebp1c-dependent transcription of key lipogenic genes to cause hepatic steatosis (Li 2010; Czech 2013). L-Rbpj livers show higher insulin sensitivity, with higher Akt phosphorylation (FIG. 15A) (Pajvani 2011). Conversely, we noted repressed phosphorylation of canonical mTORC1 targets, p70 S6 kinase and 4E-BP1 (Gingras 1999; Chiang 2005; Weng 1998), in liver and in primary hepatocytes (FIG. 15A, FIG. 15B). These data suggest that Notch is required for maximal hepatocyte mTORC1 activity.

Hepatic Notch Activity is Associated with Steatohepatitis, Independent of Insulin Resistance and More Strongly than Hepatic Lipid Content.

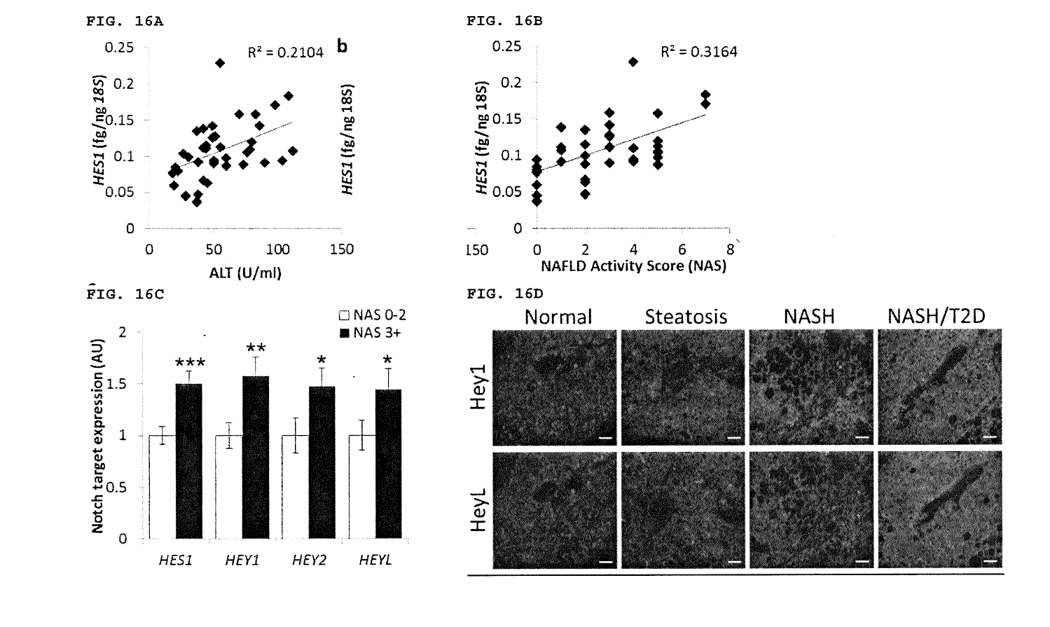

Our mouse data prompted us to study the effects of obesity on hepatic Notch signaling in people. In a cross-sectional study in human patients undergoing outpatient liver biopsy, we found that hepatic Notch activity (as assessed by HES1 expression) was positively correlated with insulin resistance, as well as hepatic triglyceride content, but showed greatest correlation with NAFLD activity score (NAS) and plasma aminotransferase (ALT) levels, which are independent predictors of progression to NASH (FIG. 16A, FIG. 16B). Similar correlation was seen with HEY1 and HEYL (not shown). As such, patients with NAS scores of 3+, correlating with high risk of NASH (Kleiner 2005; Sanyal 2011), have increased HES/HEY expression (FIG. 16C). Interestingly, although we found highest Notch activity in patients with both T2D and NASH (FIG. 16D), HES1 was significantly associated with NAS independent of either plasma insulin levels or HOMA-IR, a marker of insulin resistance (Valenti 2013).

### Dietary Models of Mouse “NASH”

These data suggested that increased hepatic Notch signaling is more associated with hepatocyte damage than simple lipid content, but we could not interpret causality. To show that Notch may provide a mechanistic link between hepatic steatosis and NASH, we needed a robust animal model of steatohepatitis. Although HFD-feeding induces marked hepatic steatosis, with lipid content approaching 20% on a gram per gram basis, this does not translate to the hepatocyte injury seen in human NASH, or the fibrotic reaction seen in cirrhotic livers. The most frequently used model for mouse “NASH” is methionine and choline deficient diets (MCDD) (Rinella 2004)—MCDD-fed mice develop macrovesicular steatosis, lobular inflammation and fibrosis (Kohli 2011). Unlike human NASH, however, MCDD-fed animals tend to lose weight and, as such, are more insulin-sensitive than control animals (Pinella 2008). This contradiction with the human condition has prompted many investigators to change dietary models, to a high-fat, high-carbohydrate diet supplemented with fructose-containing drinking water (henceforth, HFHC diet) (Kohli 2011; Kohli 2010). HFHC-feeding induces obesity, and similar insulin resistance and hepatic steatosis as HFD-fed animals (Kohli 2010). Unlike HFD, however, HFHC-feeding results in significant hepatocellular injury (leading to increased hepacocyte TUNEL staining and plasma ALT levels) and inflammation, as measured by immunohistochemical (F4/80 staining) and qPCR evaluation for Kupffer (resident liver macrophage) number (Emr1, Cd68) and activity (Mcp1, Tnfa). In addition, HPHC-feeding induces increase in hepatic stellate cell (HSC) number and activity, which leads to fibrosis with elevated collagen expression and Masson's trichrome staining (Ibrahim 2013). Thus, HFHC-feeding reproduces all three key aspects of NASH—obesity/insulin-resistance, hepatocyte injury and inflammation/fibrosis—and is the current state of the art in modeling human NASH in mice.

### Both MCDD- and HFHC-Feeding Induce Hepatic Notch Activity

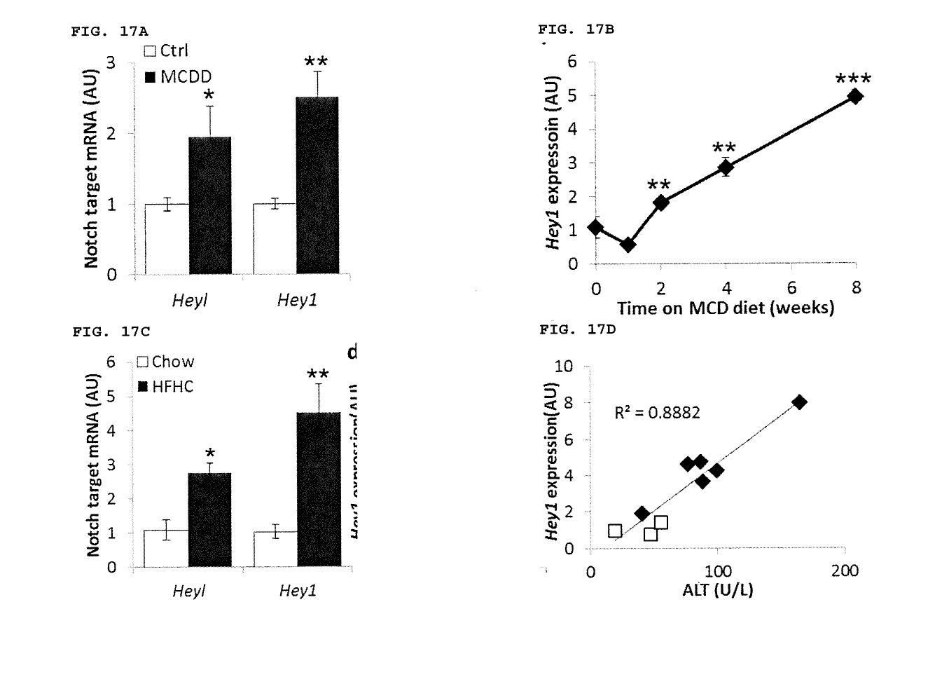

Although we show that liver Notch activity is higher in patients with NASH (Valenti 2013), we did not know whether MCDD and HFHC-feeding would similarly be associated with higher Notch signaling. Interestingly, in a series of collaborative efforts, we found that both diets strongly induced hepatic Notch activity in wildtype mice. First, we looked at a cohort of adult, wildtype mice sacrificed after 2 weeks of MCDD feeding (Chen 2008), and found a ˜2-3 fold increase in liver Notch activity (FIG. 17A). In a separate cohort of wildtype mice, sacrificed every 2 weeks after initiation of MCDD (Clarke 2014), we found a time-dependent further increase in Notch activity (FIG. 17B). Given the limitations of the MCDD as discussed above, we next determined the effects of HFHC-feeding on Notch function. Wildtype mice allowed ad libitum access to HFHC diet for 16 weeks become obese, insulin-resistant and, as reported (Kohli 2010), show pathologic features of steatohepatitis (not shown). We found HFHC-feeding significantly increased hepatic Notch activity (FIG. 17C), which showed remarkable correlation to plasma ALT levels (FIG. 17D). These data suggest that Notch activity in liver is associated with hepatocyte injury induced by two different “RASH”-provoking diets.

### Notch is a Novel “Druggable Target” for Both T2D and NAFLD/NASH.

Despite its clear importance in regulation of hepatic insulin sensitivity, FoxO1 is a poor drug target due to its nuclear location and the lack of a ligand-binding domain. Similarly, available mTOR inhibitors have multiple liabilities, including non-specificity to the two mTOR-containing complexes (mTORC1 and mTOPC2) (Lamming 2012) that have distinct functions (Lamming 2013), resulting in metabolic side-effects including glucose intolerance and dyslipidemia (Blattler 2012; Almeida 2013). In contrast, Notch signaling is therapeutically accessible, given its plasma membrane location, well-defined ligand-binding domain and equally well-characterized downstream signaling cascade. Simultaneous improvement in whole-body glucose and lipid metabolism in the absence of altered weight or adiposity, resulting in reduced atherosclerosis, is quite rare, and suggests Notch as a novel means of reducing metabolic disease burden as obesity rates continue to rise.

### Notch Decoy Prevents Diet-Induced Glucose Intolerance and Fatty Liver.

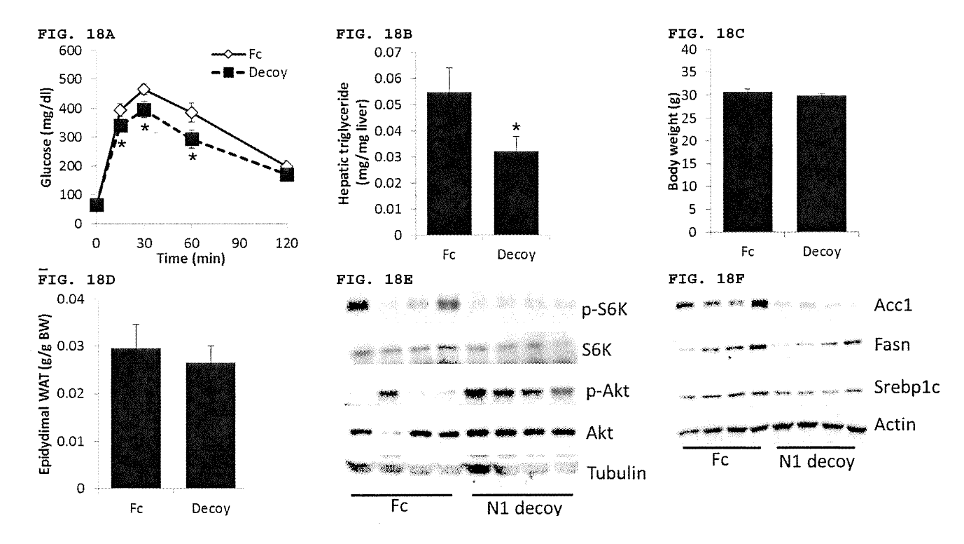

To confirm our findings in the L-Rbpj mouse model, we tested whether acute inhibition of Notch signaling with Notch1 decoy receptor (henceforth, Decoy) that encodes only the extracellular domain (Funahashi 2008; Funahashi 2011) and acts in a dominant-negative manner by sequestering endogenous ligand, can similarly protect from diet-induced glucose intolerance and fatty liver. Consistent with results from L-Rbpj mice, Notch decoy administration to HFD-fed mice improved glucose tolerance (FIG. 18A) and lowered liver TG (FIG. 18B). Body weight and adiposity were unaffected (FIG. 18C, FIG. 18D), suggesting the metabolic improvements in Decoy-treated mice were mediated through weight-independent effects in the liver. In fact, similar to L-Rbpj liver, Decoy-treated mice showed higher liver insulin sensitivity with a parallel reduction in mTorc1 activity (FIG. 18E), leading to reduced lipogenic protein expression (FIG. 18F). Beyond a proof of principle of therapeutic potential of Notch inhibition, these data suggest that Notch ligand-specific inhibitors will reap similar metabolic benefits without known side-effects of non-specific Notch inhibitors (Milano 2004; van Es 2005).

### Jagged1 is the Primary Notch Ligand in Liver.

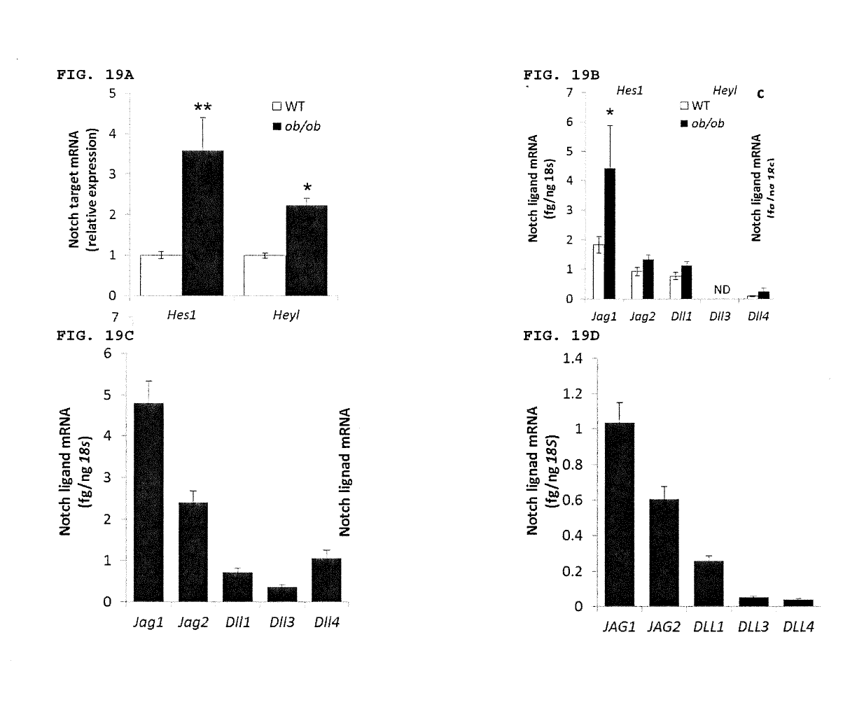

We next hypothesized that specific inhibition of only the relevant Notch ligand in liver may show the same benefit while minimizing potential side-effects. By differential centrifugation after collagenase perfusion, we can achieve near-100% purity of hepatocytes and non-hepatocyte fractions from whole liver. Hepatocytes isolated from obese, insulin-resistant DIO or ob/ob mice show increased Notch activity (not shown and FIG. 19A), and a proportionate increase in expression of Jagged1 (Jag1); we observe no meaningful change in other ligands or any ligand in the non-hepatocyte fraction (FIG. 19B, and not shown). This is consistent with recent data demonstrating that Jag1 expression in endothelial cells is increased in hyperglycemia, resulting in altered normal Notch-mediated angiogenesis, an effect recapitulated with Jagged1 overexpression or rescued with shRNA to Jagged1 (Yoon 2013). Of note, Jag1 is expressed at 2-3× higher levels in mouse and human liver than the next most abundant ligand, Jag2 (FIG. 19C, FIG. 19D).

Jagged-Specific Inhibition with the Notch Decoy Variant, N1d₁₉₋₂₄, Reduces Gluconeogenic and Lipogenic Gene Expression.

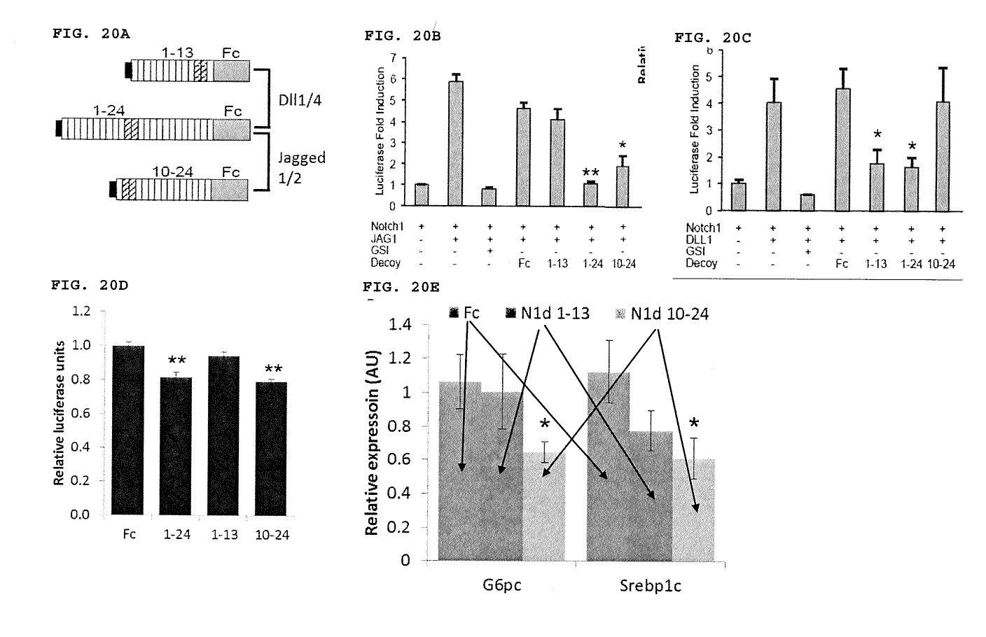

Our group has developed Decoy variants N1d₁₋₁₃ and N1d₁₀₋₂₄, based on number of EGF repeats, which block Notch signaling in a ligand-specific manner (FIG. 20A). Whereas parent Notch decoy blocks both Jagged1/2 and D11-1/4-mediated Notch signaling, N1d₁₋₁₃ only inhibits D11-1/4-induced and N1d₁₀₋₂₄ only Jagged1/2-induced signaling (FIG. 20B, FIG. 20C). We transfected hepatoma cells with a Notch-responsive luciferase reporter, then added Notch-decoy transfected and secreted (“conditioned media”) from HEK 293 cells. We found that either parent Decoy or N1d₁₀₋₂₄ conditioned media, but not N1d₁₋₁₃, blocked endogenous Notch activity (FIG. 20D). This critical experiment suggests that hepatocyte-hepatocyte interactions are sufficient to activate Notch signaling in vitro, and that this signal likely arises from Jagged ligands.

We next tested whether N1d₁₀₋₂₄ would block gluconeogenic and lipogenic gene expression. We transduced obese mice with Fc (control), N1d₁₋₁₃ or N1d₁₀₋₂₄ adenoviruses. After sacrifice, we isolated livers, and prepared liver cDNA for quantitative PCR of these critical Notch targets. Nice transduced with N1d₁₀₋₂₄, but not control or Delta-like-specific adenovirus, showed decreased obesity-induced activation of G6pc and Srebp1c expression (FIG. 20E). Of note, the level of inhibition of these key gluconeogenic and lipogenic genes was similar to what was previously seen with parent Decoy (not shown). This result suggests that Jagged-specific inhibition of hepatic Notch signaling is sufficient to protect from obesity-related metabolic complications, and opens up the possibility of a safe and effective Notch antagonist for treatment of T2D and NAFLD/NASH.

### SUMMARY

Obesity is associated with T2D and NAFLD/NASH, as well as increased Notch activity in liver in mice and humans.
Gain-of-function studies in mice prove that inappropriate activation of Notch leads to T2D and NAFLD, and may provide the molecular bridge by which simple steatosis leads to NASH. Notch signaling is ideally positioned to mediate cellular cross-talk.
Genetic Notch loss-of-function mouse models prove that Notch is necessary for the full development of obesity-induced T2D and NAFLD/NASH.
Pharmacologic inhibitors of Notch signaling, including Notch decoys, monoclonal antibodies to Notch receptors/ligands or small-molecule inhibitors, originally developed for cancer, may be repurposed as a therapy for the rapidly expanding patient population with T2D and NAFLD/NASH.

### REFERENCES FOR SECOND SERIES OF EXPERIMENTS

Almeida, C. C., ec al. Safety of immunosuppressive drugs used as maintenance therapy in kidney transplantation: a systematic review and meta-analysis. Pharmaceuticals (Basel) 6, 1170-1194 (2013).
Blattler, S. M., et al. Yin Yang 1 deficiency in skeletal muscle protects against rapamycin-induced diabetic-like symptoms through activation of insulin/IGF signaling. Cell metabolism 15, 505-517 (2012).
Bolos, V., Grego-Bessa, J. & de la Pompa, J. L. Notch signaling in development and cancer. Endocrine reviews 28, 339-363 (2007).
Bugianesi, E., et al. Insulin resistance in non-diabetic patients with non-alcoholic fatty liver disease: sites and mechanisms. Diabetologia 48, 634-642 (2005).
Chan, S. M., Weng, A. P., Tibshirani, R., Aster, J. C. & Utz, P. J. Notch signals positively regulate activity of the mTOR pathway in T-cell acute lymphoblastic leukemia. Blood 110, 278-286 (2007).
Chiang, G. G. & Abraham, R. T. Phosphorylation of mammalian target of rapamycin (mTOR) at Ser-2448 is mediated by p70S6 kinase. The Journal of biological chemistry 280, 25485-25490 (2005).
Clarke, J. D., et al. Circulating microRNA 122 in the methionine and choline-deficient mouse model of non-alcoholic steatohepatitis. J Appl Toxicol 34, 726-732 (2014).
Czech, M. P., Tencerova, M., Pedersen, D. J. & Aouadi, M. Insulin signalling mechanisms for triacylglycerol storage. Diabetologia 56, 949-964 (2013).
Efferson, C. L., et al. Downregulation of Notch pathway by a gamma-secretase inhibitor attenuates AT/mammalian target of rapamycin signaling and glucose uptake in an ERBB2 transgenic breast cancer model. Cancer research 70, 2476-2484 (2010).
Engelking, L. J., at al. Overexpression of Insig-1 in the livers of transgenic mice inhibits SREBP processing and reduces insulin-stimulated lipogenesis. The Journal of clinical investigation 113, 1168-1175 (2004).
Funahashi, Y., et al. A notch1 ectodomain construct inhibits endothelial notch signaling, tumor growth, and angiogenesis. Cancer research 68, 4727-4735 (2008).
Funahashi, Y., et al. Notch modulates VEGF action in endothelial cells by inducing Matrix Metalloprotease activity. Vascular cell 3, 2 (2011).
Gingras, A. C., et al. Regulation of 4E-BP1 phosphorylation: a novel two-step mechanism. Genes & development 13, 1422-1437 (1999).
Haeualer, R. A., Pratt-Hyatt, M., Welch, C. L., Klaassen, C. D. & Accili, D. Impaired generation of 12-hydroxylated bile acids links hepatic insulin signaling with dyslipidemia. Cell metabolism 15, 65-74 (2012).
Horton, J. D., Goldstein, J. L. & Brown, M. S. SREBPs: transcriptional mediators of lipid homeostasis. Cold Spring Harbor symposia on quantitative biology 67, 491-498 (2002).
Ibrahim, S. H., et al. Mixed lineage kinase 3 deficient mice are protected against the high fat high carbohydrate diet-induced steatohepatitis. Liver Inc (2013).
Kahn, S. E., et al. Glycemic durability of rosiglitazone, metformin, or glyburide monotherapy. The New England journal of medicine 355, 2427-2443 (2006).
Kim, J. B., Wright, H. N., Wright, N. & Spiegelman, B. M. ADD1/SREBP1 activates PPARgamma through the production of endogenous ligand. Proceedings of the National Academy of Sciences of the United States of America 95, 4333-4337 (1998).
Kitamura, T., et al. A Foxo/Notch pathway controls myogenic differentiation and fiber type specification. The Journal of clinical investigation 117, 2477-2485 (2007).
Kleiner, D. E., et al. Design and validation of a histological scoring system for nonalcoholic fatty liver disease. Hematology 41, 1313-1321 (2005).
Kohli, R. & Feldstein, A. E. NASH animal models: are we there yet? Journal of hepatology 55, 941-943 (2011).
Kohli, R., et al. High-fructose, medium chain trans fat diet induces liver fibrosis and elevates plasma coenzyme Q9 in a novel murine model of obesity and nonalcoholic steatohepatitis. Hepatology 52, 934-944 (2010).
Laming, D. W. & Sabatini, D. M. A Central role for mTOR in lipid homeostasis. Cell metabolism 18, 465-469 (2013).
Lamming, D. W., et al. Rapamycin-induced insulin resistance is mediated by mTORC2 loss and uncoupled from longevity. Science 335, 1638-1643 (2012).
Li, S., Brown, M. S. & Goldstein, J. L. Bifurcation of insulin signaling pathway in rat liver: mTORC1 required for stimulation of lipogenesis, but not inhibition of gluconeogenesis. Proceedings of the National Academy of Sciences of the United States of America 107, 3441-3446 (2010).
Liang, G., et al. Diminished hepatic response to fasting/refeeding and liver X receptor agonists in mice with selective deficiency of sterol regulatory element-binding protein-1c. The Journal of biological chemistry 277, 9520-9528 (2002).
Lin, H. V. & Accili, D. Hormonal regulation of hepatic glucose production in health and disease. Cell metabolism 14, 9-19 (2011).
Matsuda, N., et al. SREBP cleavage-activating protein (SCAP) is required for increased lipid synthesis in liver induced by cholesterol deprivation and insulin elevation. Genes & development 15, 1206-1216 (2001).
Matsumoto, M., Han, S., Kitamura, T. & Accili, D. Dual role of transcription factor FoxO1 in controlling hepatic insulin sensitivity and lipid metabolism. J Clin Invest 116, 2464-2472 (2006).
Matsumoto, H., Pocai, A., Rossetti, L., Depinho, R. A. & Accili, D. Impaired regulation of hepatic glucose production in mice lacking the forkhead transcription factor Foxo1 in liver. Cell metabolism 6, 208-216 (2007).
Milano, J., et al. Modulation of notch processing by gamma-secretase inhibitors causes intestinal goblet cell metaplasia and induction of genes known to specify gut secretory lineage differentiation. Toxicological sciences: an official journal of the Society of Toxicology 82, 341-358 (2004).
Millar, J. S., Cromley, D. A., McCoy, M. G., Rader, D. J. & Billheimer, J. T. Determining hepatic triglyceride production in mice: comparison of poloxamer 407 with Triton WR-1339. Journal of lipid research 46, 2023-2028 (2005).
Oka, C. & Kawaichi, M. V(D)J recombination of immunoglobulin genes. Advances in biophysics 31, 163-180 (1995).
Oka, C., et al. Disruption of the mouse RBP-J kappa gene results in early embryonic death. Development 121, 3291-3301 (1995).
Pajvani, U. B., et al. Inhibition of Notch signaling ameliorates insulin resistance in a FoxO1-dependent manner. Nature medicine 17, 961-967 (2011).
Pajvani, U. B., et al. Inhibition of Notch uncouples Akt activation from hepatic lipid accumulation by decreasing mTorc1 stability. Nature medicine 19, 1054-1060 (2013).
Postic, C. & Girard, J. Contribution of de novo fatty acid synthesis to hepatic steatosis and insulin resistance: lessons from genetically engineered mice. The Journal of clinical investigation 118, 829-838 (2008).
Postic, C. & Magnuson, N. A. DNA excision in liver by an albumin-Cre transgene occurs progressively with age. Genesis 26, 149-150 (2000).
Rametta, P., et al. Increased insulin receptor substrate 2 expression is associated with steatohepatitis and altered lipid metabolism in obese subjects. Int J Obes (Lond) (2012).
Rinella, M. E. & Green, R. N. The methionine-choline deficient dietary model of steatohepatitis does not exhibit insulin resistance. Journal of hepatology 40, 47-51 (2004).
Rinella, N. E., et al. Mechanisms of hepatic steatosis in mice fed a lipogenic methionine choline-deficient diet. Journal of lipid research 49, 1068-1076 (2008).
Sabatini, D. M. mTOR and cancer: insights into a complex relationship. Nature reviews. Cancer 6, 729-734 (2006).
Sanyal, A. J., et al. Endpoints and clinical trial design for nonalcoholic steatohepatitis. Hepatology 54, 344-353 (2011).
Savage, D. B. A Semple. R. K. Recent insights into fatty liver, metabolic dyslipidaemia and their links to insulin resistance. Current opinion in lipidology 21, 329-336 (2010).
Shimomura, I., et al. Insulin selectively increases SREBP-1c mRNA in the livers of rats with streptozotocin-induced diabetes. Proceedings of the National Academy of Sciences of the United States of America 96, 13656-13661 (1999).
Swiatek, P. J., Lindsell, C. E., del Amo, F. F., Weinmaster, G. & Gridley, T. Notch1 is essential for postimplantation development in mice. Genes & development 8, 707-719 (1994).
Tao, R., et al. Hepatic FoxOs regulate lipid metabolism via modulation of expression of the nicotinamide phosphoribosyltransferase gene. The Journal of biological chemistry 286, 14681-14690 (2011).
Valenti, L., et al. Hepatic Notch Signaling Correlates with Insulin Resistance and Non-Alcoholic Fatty Liver Disease. Diabetes (2013).
van Es, J. H., et al. Notch/gamma-secretase inhibition turns proliferative cells in intestinal crypts and adenomas into goblet cells. Nature 435, 959-963 (2005).
Villanova, N., et al. Endothelial dysfunction and cardiovascular risk profile in nonalcoholic fatty liver disease. Hepatology 42, 473-480 (2005).
Wang, Q. P., et al. Regulation of the p70 S6 kinase by phosphorylation in vivo. Analysis using site-specific anti-phosphopeptide antibodies. The Journal of biological chemistry 273, 16621-16629 (1998).
Yecies, J. L., et al. Akt stimulates hepatic SREBP1c and lipogenesis through parallel mTOPC1-dependent and independent pathways. Cell metabolism 14, 21-32 (2011).
Yilmaz, Y. Review article: is non-alcoholic fatty liver disease a spectrum, or are steatosis and non-alcoholic steatohepatitis distinct conditions? Aliment Pharmacol Ther 36, 815-823 (2012).
Yoon, C. H., et al. High glucose-induced jagged 1 in endothelial cells disturbs notch signaling for angiogenesis: A novel mechanism of diabetic vasculopathy. J Mol Cell Cardiol (2013).

What is claimed:

1. A method of treating a subject suffering from a fatty liver disease which comprises administering to the subject an amount of a Notch1 decoy protein in an amount effective to treat the subject's fatty liver disease.

2. The method of claim 1, wherein the fatty liver disease is alcoholic fatty liver disease.

3. The method of claim 1, wherein the fatty liver disease is non-alcoholic fatty liver disease.

4. The method of claim 1, wherein the fatty liver disease is non-alcoholic steatohepatitis.

5. The method of claim 1, wherein the subject is also suffering from metabolic syndrome.

6. The method of claim 1, wherein the subject is also suffering from diabetes.

7. The method of claim 1, wherein the subject is also suffering from hypertension.

8. The method of claim 1, wherein the subject is also suffering from obesity.

9. The method of claim 1, wherein the subject is also suffering from dyslipidemia.

10. The method of claim 1, wherein the Notch1 decoy protein comprises (a) amino acids, the sequence of which is identical to the sequence of a portion of the extracellular domain of a human Notch1 receptor protein and (b) amino acids, the sequence of which is identical to the sequence of an Pc portion of an antibody.

11. The method of claim 10, wherein the Pc portion of the antibody is the Fc portion of a human antibody.

12. The method of claim 10, wherein (b) is located to the carboxy terminal side of (a).

13. The method of claim 10 further comprising a linker sequence between (a) and (b).

14. The method of claim 10, wherein the portion of the extracellular domain of the human Notch1 receptor protein is selected from the group consisting of Notch1 EGF-like repeats 1-36, Notch1 EGF-like repeats 1-13, Notch1 EGF-like repeats 1-24, Notch1 EGF-like repeats 9-23, Notch1 EGF-like repeats 10-24, Notch1 EGF-like repeats 9-36, Notch1 EGF-like repeats 10-36, Notch1 EGF-like repeats 14-36, Notch1 EGF-like repeats 13-24, Notch1 EGF-like repeats 14-24, Notch1 EGF-like repeats 25-36.

15. The method of claim 14, wherein the portion of the extracellular domain of the human Notch1 receptor protein is Notch1 EGF-like repeats 1-24.

16. The method of claim 14, wherein the portion of the extracellular domain of the human Notch1 receptor protein is Notch1 EGF-like repeats 10-24.

17. The method of claim 14, wherein the portion of the extracellular domain of the human Notch receptor protein is Notch1 EGF-like repeats 1-36.

18. The method of claim 1, wherein the Notch decoy protein only inhibits Jagged-induced signaling.

19. The method claim 1, wherein treating comprises reducing hepatic triglycerides.

20. The method claim 1, wherein the Notch decoy protein is administered in connection with a diet regimen.

21. The method claim 1, wherein the Notch decoy protein is administered in connection with an exercise regimen.

22. The method claim 1, wherein the Notch decoy protein is administered as a monotherapy.

23. The method claim 1, wherein the Notch1 decoy protein is administered in combination with one or more additional agents for the treatment of the fatty liver disease.

24. The method of claim 23, wherein the one or more additional agents for the treatment of the fatty liver disease are selected from the group consisting of vitamin 3, selenium, betadine, metformin, rosiglitazone, pioglitazone, insulin sensitizers, antioxidants, probiotics, Omega-3 DHA, pentoxifylline, anti-TNF-alpha, FXR agonists and GLP-1 agonists.

25. A composition comprising a pharmaceutically acceptable carrier and an amount of a Notch1 decoy protein effective to treat a fatty liver disease.

26. A package comprising:
(a) the pharmaceutical composition of claim 25; and
(b) instructions for using the pharmaceutical composition of step (a) to treat the fatty liver disease.

27. A method of treating a subject suffering from a fatty liver disease which comprises administering to the subject a Jagged inhibitor in an amount effective to treat the subject's fatty liver disease.

28. A composition comprising a pharmaceutically acceptable carrier and an amount of a Jagged inhibitor effective to treat a fatty liver disease.
